(19) United States
(12) Patent Application Publication
(10) Pub. No.: US 2012/0130340 A1
(43) Pub. Date: May 24, 2012

(54) Tissue Desensitization Instrument & Method

(76) Inventor: Eric J. Knutson, (US)

(21) Appl. No.: 12/949,792

(22) Filed: Nov. 19, 2010

(51) Int. Cl. A61M 5/42

(52) U.S. Cl. 604/506; 604/112

(57) ABSTRACT

An automatic fluid injector (10, 66, or 76) having a coolant-absorbent pad (24) for pressing onto a tissue site for desensitization. When the injector (10, 66, 76) is pressed further toward the tissue site, the contained fluid is pressurized, and an enclosed needle (16) moves distally to penetrate a distal wall of injector (10, 66, 76). While needle (16) is being repositioned distally, the pressurized fluid is blocked from flowing into needle (16). The fluid blockage prevents the fluid from inadvertently warming the cold, desensitizing pad (24) and causing the patient to experience pain during needle (16) penetration. When needle (16) penetrates to a preset depth, and is sealed in the tissues, then the pressurized fluid is unblocked, and flows automatically through needle (16). In another embodiment, an instrument (46) having a coolant-absorbent felt (52) is able to unobstructably desensitize tissues prior to penetration with sharps.

### BACKGROUND

1. Field of Invention

This invention relates to medicine and dentistry, specifically to desensitization of tissues required in association with traumas.

2. Description of Prior Art

In medicine and dentistry, tissues are frequently subjected to traumas, such as periodontal ligament injections, intraosseous injections, general tissue injections, drawing blood, glucose tests, biopsies, lancing abscesses, and so on. Typically the tissues involved are the skin or mucosa epithelial and subepithelial tissues. However, the periosteum, and other tissues may also be involved.

For the descriptions herein, an instrument causing any trauma is called a sharp, and traumas are called punctures. Sharps include needles, aspirators, scalpels, biopsy punches, biopsy brushes, intraosseous perforators, lancets, lasers, and so on. Traumas include punctures, burns, abrasions, and other skin irritations.

There are several methods of desensitizing tissues prior to puncture. These methods include the use of topical chemical anesthetics, Transcutaneous Electrical Nerve Stimulation (TENS), pressure, vibration, cooling, and so on.

A first method of desensitizing involves applying and removing the desensitizing means from the puncture area immediately prior to the puncture. Examples include the use of DentiPatch (Noven) anesthetic patches, pressing ice or a cold instrument to pre-cool the site, and devices of U.S. Pat. Nos. 5,639,238, 5,839,895, 5,873,844 and US Pat Appl 2006/0217636. With anesthetic patches, substantial time is required. With the ice or cold instruments, the method is somewhat awkward.

A second method of desensitizing involves applying cold, vibration, pressure, or other desensitizing means along one side of the puncturing site immediately prior or during the puncture. Examples include pressing on the tissues with a blunt instrument during the puncture, such as a dental mirror handle or a Pressure Anesthesia Device (U.S. Pat. No. 5,171,225).

A third method of desensitizing involves applying pressure to tissues substantially surrounding the puncture area immediately prior to and during the puncture. For example, pressure is maintained on the tissues with a Palatal Anesthesia Device (U.S. Pat. No. 5,088,925) while inserting a needle into the central lumen of the device.

A fourth method of desensitizing involves applying negative pressure to tissues prior and during puncture (U.S. Pat. No. 2,945,496).

A fifth method involves cooling the puncture area prior to puncturing the tissues. A first cooling method involves directing a vapocoolant aerosol spray onto the puncture area prior to a puncture. An example is Freeze aerosol spray (Hagar Worldwide). To avoid frostbite, only moderately cold vapocoolants may be used when spraying directly onto the tissues. When coolants are sprayed directly on the tissues, all the coolant evaporates before the tissue stops cooling, and begins to warm. The user has reduced control over the termination of tissue cooling. Extended cooling time increases frostbite risk at effective desensitizing temperatures. A second cooling method involves applying the cold side of a Peltier electrode to the puncture area prior to a puncture.

A sixth method involves placing TENS electrodes near the puncture area and applying current during the puncture (U.S. Pat. No. 5,496,363).

A seventh method of desensitizing involves vibrating the sharp during the puncture (U.S. Pat. Nos. 5,401,242, 5,647,851). For example, a VibraJect (VibraJect LLC) is connected to vibrate a syringe during an injection to activate a pain-gate response (U.S. Pat. No. 6,602,229).

An eighth method of desensitizing involves vibrating the tissues adjacent to the puncture area (U.S. Pat. Nos. 2,258,857, 3,620,209, 6,231,531, & EP1535572).

A ninth method of desensitizing involves applying topical anesthetic gels or liquids to the tissue for a substantial time, and puncturing the tissue through the residual anesthetics.

A tenth method of desensitizing involves stretching the puncture area (US Pat Appl 2006/0211982).

An eleventh method of desensitizing involves pinching the skin surrounding the puncture area (EP1535572)

A twelfth method of desensitizing involves applying heat to the puncture area prior and during the puncture (US Pat Appl 2006/0217636).

A thirteenth method of desensitizing involves applying cold to a tissue area prior and during the puncture with a cooled non-absorbent surface (U.S. Pat. No. 5,236,419, US Pat Appl 2006/0106363). Such a non-absorbent cooling surface tends to cool only marginally when a vapocoolant is applied. This is because a non-absorbent cooling surface does not accelerate the evaporation of the vapocoolant. Further, a non-absorbent cooling surface tends to have substantial heat capacity that competes with the tissue-cooling effect of the vapocoolant.

Application of a pre-cooled non-absorbent heat-reservoir is awkward, inconvenient, costly, or impractical for the user.

Further, the degree of cooling is difficult to control. These methods of cooling a non-absorbent surface either tend to under-cool or over-cool the surface. As a result, the tissues are either insufficiently desensitized, or they are at risk of tissue sloughing from frostbite.

This issue is compounded because the method and device causes the cooling surface to remain in contact with the tissue longer that the entire time the sharp is in the tissues. The cooled surface contacts the tissue prior to puncture by the sharp, and is not withdrawn until after the sharp is withdrawn. The tissue contact time is thereby extended. An extended tissue contact time limits the degree of cooling achievable without risk of frostbite. Warmer, less effective, coolants must therefore be used.

The cooled surface entirely surrounds the puncture point prior to the sharp entering the tissues. As such, the user is unable to visually see the sharp as it contacts and punctures the tissues.

A fourteenth method of desensitizing involves applying cold to an area surrounding a puncture point prior and during puncture with an absorbent surface, such as a felt, and an evaporative coolant (US Pat Appl 2009/0004628-A1). The felt is shown supported by a disc at the end of a linear hand instrument, such as a mouth mirror.

The above tissue desensitization methods suffer from one or more of a number of disadvantages:
(a) Inadequate desensitization
(b) Method requires excessive time
(c) Method is inconvenient
(d) Obstructed view of sharp penetrating tissue
(e) Obstructed access of sharp to tissue site
(f) Needle sharps unable to enter tissue from a low angle
(g) Effective desensitization performed at risk of frostbite
(h) Fluid injection step manually performed

### SUMMARY OF THE INVENTION

The invention is a device and method for desensitizing tissues. More particularly, the tissues are desensitized to permit the comfortable insertion of a sharp.

For example, the device desensitizes the tissues, comfortably inserts a needle, and injects a few drops of fluid. The fluid may comprise a local anesthetic used to lightly anesthetize the tissues prior to a minor procedure, such as a higher-volume local anesthetic injection, an intraosseous anesthesia drill, minor cryosurgery, or a minor scalpel procedure. The fluid may also comprise an allergen, a dermal filler, a dye, and so on.

In a typical embodiment, the device comprises a hand instrument having a precharged fluid medicament capsule at the distal end thereof. The capsule contains a given volume of fluid and a needle. The capsule walls are comprised of an elastomeric material.

The distal end of the capsule has a disc for pressing against the tissue site to be anesthetized. The disc has a receiver side and a tissue side. The disc receiver side is for receiving the needle. The receiver side is configured to guide the needle toward a slot opening through the disc. In use, the needle is directed through the slot en route to puncturing a puncture point in the tissues.

The tissue side of the disc has a coolant absorbent surface. When the absorbent surface has absorbed a vapocoolant, the evaporating vapocoolant cools the absorbent surface. When the cold absorbent surface is pressed onto the tissue site, the absorbent surface substantially surrounds or covers the puncture point. The cold absorbent surface thereby substantially cools the puncture point, thereby desensitizing the tissues prior to a puncture.

In use, the user applies vapocoolant to the coolant absorbent surface. The vapocoolant evaporates such that the absorbent surface is made cold, and appears frosty. The cold absorbent surface is pressed onto the tissue site. The tissue site is made cold by contact with the cold absorbent surface. The cold tissue is substantially desensitized for subsequent puncture.

The user further grasps a handle of the instrument in order to compress the capsule against the tissue site. The handle, or extension of the handle, also pushes against the distal end of the needle within the capsule. The needle is pushed through the slot of the disc, through the absorbent surface, and punctures the tissue site. The patient primarily senses only the cold, and does not experience significant discomfort.

The fluid in the capsule is pressurized by the handle extension pressing against the proximal end of the capsule. The capsule walls distend somewhat in accommodation to the fluid pressure. As the capsule walls distend, the extension pushes the needle into the tissues. Ports in the side of the needle are located such that they are brought in fluid communication with the capsule fluid, but only after the needle has entered the tissues to a specified depth. Once the needle penetrates the tissues to a sufficient depth, the pressurized fluid automatically moves through the ports, into the needle, and into the tissues. Once sufficient fluid has entered the tissues, user withdraws the handle extension away from the tissues.

In economical embodiment, the capsule may have no distal disc. Instead, the walls of the capsule extend over the distal area to form a distal capsule wall. The'needle penetrates directly through the distal capsule wall.

In another economical embodiment, the tissues are desensitized utilizing a disc having no pre-charged capsule. The disc is connected directly to a hand instrument. User provides a syringe and needle to deliver a fluid through the slot in the disc and into the tissues. The disc affords substantially unobstructed visual and sharps access.

The invention comprises an automatic injector having a tissue desensitizing, coolant-absorbent surface that substantially encompasses a needle protrusion area. The injector has a protrudable needle initially located in a nonprotruded position. An unblockable fluid blocking means, a fluid blocking means that is capable of being unblocked, initially blocks the fluid from flowing through the needle. The injector includes a fluid container that is able to pressurize the fluid to form a pressurized fluid. The fluid is pressurized when pressure is exerted against the injector by pressing the injector against the tissues. The needle is repositionable to a protruded position by pressure exerted against the injector. When in the protruded position, the needle has been repositioned a sufficient distance from the initial non-protruded position such that the fluid blocking means is unblocked. Unblocking the fluid blocking means permits automatic fluid flow through the needle, but only after said needle has sealingly penetrated into the tissues.

According to another aspect, the invention provides a tissue desensitizing puncture method comprising the steps of: desensitizingly pressing a cooled, absorbent surface of an automatic, needle-containing, fluid-containing, injector onto the tissues, thereby pressurizing the fluid to form a pressurized fluid, wherein the pressurized fluid is initially blocked from flowing through the needle, protruding the needle from the injector such that the needle sealingly penetrates the tissues, and unblocking the pressurized fluid such that the pressurized fluid automatically flows through the needle and into the tissues.

The invention provides another tissue desensitizing puncture method comprising the steps of: unobstructively cooling a puncture point by substantially encompassing the puncture point with a substantially unobstructive, cold, coolant-absorbent instrument, wherein the instrument substantially does not block visual or sharps access to the puncture point, puncturing the puncture point with a sharp, removing the instrument from the tissue, and withdrawing the sharp from the puncture point.

### OBJECTS AND ADVANTAGES

Accordingly, several objects and advantages of my invention are to provide effective tissue desensitization:
(a) that permits unobstructed visual contact with a puncture point
(b) that provides an unobstructed pathway for a sharp to a puncture point
(c) wherein sharps may enter a tissue site from a convenient and effective angle
(d) where injection fluids do not inadvertently warm a tissue-coolant surface
(e) where pressure is concentrated toward a puncture point
(f) rapidly
(g) without substantial tissue damage

Further objects and advantages are to provide pain control of short duration, so that tissue sensation returns to normal soon after the procedure. Still further objects and advantages will become apparent from a consideration of the ensuing description and drawings.

### DRAWING FIGURES

In the drawings, closely related figures have the same number, but different alphabetic suffixes.

FIG. 1 is a cross-section view of a capsule having a disc

FIG. 2 is a cross-section view of a capsule with no disc

FIG. 3 is a cross-section view of a capsule partially compressed against a tissue site

FIG. 4 is a cross-section view of a capsule fully compressed, with needle inserted

FIG. 5 is a perspective view of a disc with no capsule

FIG. 6 is a perspective view of a needle injecting through a disc

FIG. 7A is an elevation side-view of a hand instrument with disc

FIG. 7B is a top-view of hand instrument with disc

FIG. 7C is an elevation end-view of a hand instrument with disc

FIG. 8A is a cross-section view of an uncompressed sleeve injector

FIG. 8B is a cross-section view of a compressed sleeve injector

FIG. 9A is a cross-section view of an uncompressed piston injector

FIG. 9B is a cross-section view of a compressed piston injector

FIG. 10 is a cross-section view of an uncompressed injector with a pressure valve

FIG. 11A is a cross-section view of an uncompressed injector with an internal shell

FIG. 11B is a cross-section view of a compressed injector with a ruptured internal shell

Reference Numerals in Drawings

| | |
|---|---|
| 20 injector | 22 extension |
| 24 capsule | 26 needle |
| 28 disc | 30 receiver |
| 32 slot | 34 pad |
| 36 membrane | 38 septum |
| 40 hole | 42 socket |
| 44 chamber | 46 port |
| 48 diskette | 50 purchase |
| 52 connector | 54 handle |
| 56 instrument | 58 funnel |
| 60 slit | 62 felt |
| 64 film | 66 sharp |
| 68 retractor | 70 sleeve |
| 72 recess | 74 sleeve hole |
| 76 sleeve injector | 78 piston |
| 80 cylinder | 82 stopper |
| 84 flange | 86 piston injector |
| 88 valve | 90 tube |
| 92 shell | 94 filter |

### DESCRIPTION

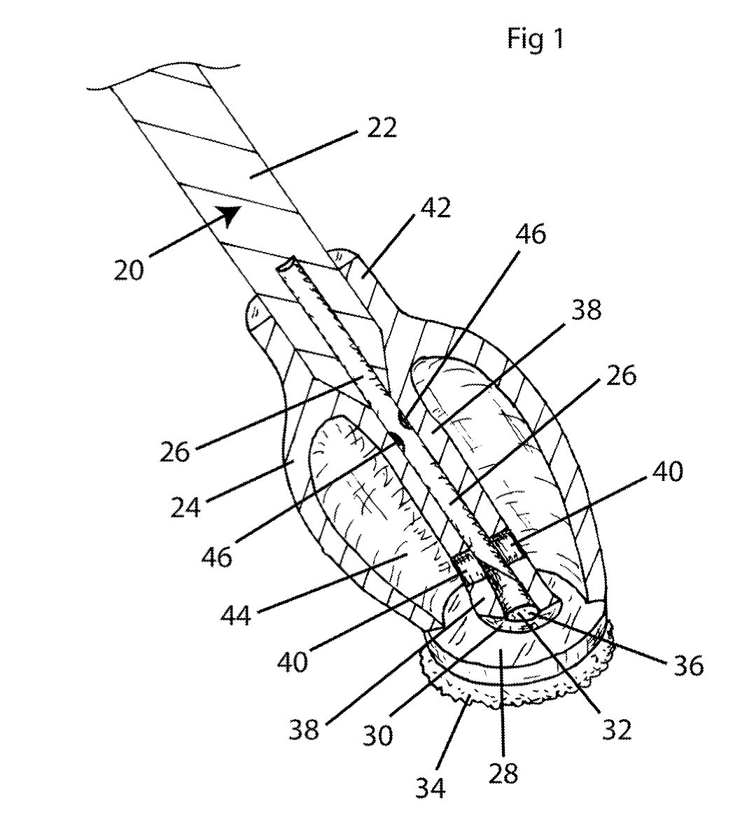

### FIGS. 1 to 9

According to one aspect, the invention provides a hand instrument for desensitizing tissues.

FIG. 1 shows an injector 20 comprising an extension 22 supporting a precharged medicament capsule 24 at the distal end of extension 22. Capsule 24 contains a given volume of medicament fluid and a needle 26. It is preferred that capsule 24 walls are comprised of an elastomeric material.

It is preferred that extension 22 is connected to a handle, whereby the handle is gripped by a user. However, extension 22 may comprise the portion of the injector 20 that is directly gripped by a user.

Extension 22 is comprised of a rigid or semi-rigid material such that user is able to exerted substantial pressure on capsule 24 by grasping and pressing with extension 22. It is preferred that extension 22 is comprised of a rigid metal, resin, or wood. However, extension 22 may be comprised of the same elastomer that comprises capsule 24. Further, extension 22 and capsule 24 may comprise a single piece casting.

The distal end of capsule 24 has a rigid disc 28 for pressing against the tissue site to be anesthetized. It is preferred that the perimeter shape of disc 28 has an ovoid geometry. It is further preferred that the bevel of needle 26 is oriented to be closest to the narrow end of disc 28. As such, a user has a visual representation of the needle 26 bevel orientation. However, disc 28 may have another geometry, especially those that may provide visual information to the user regarding the orientation of the needle 26 bevel. It is preferred that disc 28 is comprised of an injection molded plastic.

The proximal surface of disc 28 has a funnel-like receiver 30 for receiving a needle 26. Receiver 30 surrounds a central slot 32. Receiver 30 has walls that slope toward slot 32, such that when needle 26 is directed toward slot 32, but is slightly misdirected, needle 26 would tend to be deflected by receiver 30 toward slot 32.

It is preferred that slot 32 comprises an elongated hole through disc 28. However, slot 32 may comprise an elongated hole that is open on one side out to the perimeter of disc 28, may comprise a simple round hole in disc 28, and so on.

It is preferred that the diameter of slot 32 is no larger than required to permit passage of a needle 26. However, slot 32 may be much larger, such as to permit direct user visual contact with the tissue surface about a puncture point, or to permit convenient instrument access to the tissue site.

Further, slot 32 may comprise a perforatable thin area of disc 28, wherein no hole is present through disc 28. Needle 26 would perforate the thin slot 32 area of disc 28, and pass through disc 28 prior to penetrating the tissue site. Such a perforatable slot 32 enhances tissue desensitization by increasing pressure and cold applied directly to the puncture point.

The tissue side of disc 28 is covered by a coolant-absorbent surface, pad 34. Pad 34 comprises the distalmost surface of capsule 24. Pad 34 is comprised of an absorbent material, such as felt, cloth, an array of fibers, and so on. Pad 34 may comprise a separate structure from disc 28, such as an adhered pad, or pad 34 may comprise a fibrous outer layer embedded in, or adhered to, the distal surface of disc 28. Further, pad 34 may extend to cover the entire external surface of capsule 24, portions of extension 22, and so on.

Needle 26 is protrudable from injector 20. The initial position of needle 26 is a nonprotruded position. The area of the distal surface of pad 34 where needle 26 penetrates through in order to protrude beyond pad 34 is considered to comprise a needle 26 protrusion area. Pad 34 substantially encompasses the needle 26 protrusion area.

The pad 34 material has a substantially irregular surface that effectively increases the overall surface area of a given surface. A pad 34 material further provides a multiplicity of small spaces into which liquids are efficiently drawn by capillarity. A vapocoolant absorbed into a pad 34 material is therefore able to evaporate from an increased surface area. As such, the rate of evaporation of a given volume of vapocoolant is faster from a pad 34 surface relative to the rate of evaporation from a smoother surface coated with vapocoolant.

The rapid rate of evaporation from a pad 34 surface provides a substantially increased overall cooling capacity thereof. The increased cooling capacity is able to rapidly and thoroughly cool a contacted tissue site, the disk 18 tissue surface, and other liquids or materials that might come into contact with disk 18 during use, such as saliva, mucous, anesthetics, medicaments, and so on. As such, the increased cooling capacity of a pad 34 surface of disk 18 is able to effectively cool a tissue site by overpowering other reservoirs of heat that may be present in various adjacent materials. A vapocoolant evaporating from a pad 34 surface can therefore rapidly cool a tissue site, and thereby efficiently and conveniently desensitize the site.

It is preferred that the fiber density of pad 34 is somewhat reduced immediately over slot 32. A reduced pad 34 density over slot 32 would decrease the overall thickness of disc 28, further enhancing user visualization and instrument access to the tissue site. It would also reduce interference to the movement of needle 26 through pad 34 caused by the fibers of pad 34. A reduced fiber density may comprise a reduced thickness of pad 34. Further, a slot may be present in pad 34 that is alignable with slot 32. Reducing fiber density over slot 32 is intended increase, or concentrate, the direct pressure applied by disc 28 against the tissue site, to facilitate tissue desensitization. However, pad 34 may have full fiber density, or even increased fiber density over slot 32, as a means for increasing pressure exerted on the tissue site.

The tissue side of pad 34 is shown having a membrane 36 spanning across slot 32. Membrane 36 enhances puncture point cooling, especially in an area of 34 where the fibers are sparse or absent. It is preferred that membrane 36 is comprised of an absorbent, fibrous material. However, membrane 36 may be comprised of a permeable material such that vapocoolant readily penetrates through membrane 36 and is absorbed into pad 34. It is preferred that membrane 36 span only across slot 32. However, membrane 36 may span across the entire breadth of pad 34, or it may be absent.

Alternatively, membrane 36 may be located on the side of pad 34 that contacts the distal wall disc 28, between pad 34 and disc 28. In this location, membrane 36 may provide vapocoolant absorbance in the area over the puncture point where the fibers of pad 34 may be sparse. Capsule 24, disc 28, or needle 26, may push membrane 36 into contact with the tissue surface. Membrane 36 may also provide the user a tactile or audible sensation indicating that needle 26 has penetrated the distal end of capsule 24, such as by tearing, popping, and so on.

It is preferred that disc 28 and receiver 30 provide a convex supporting surface for pad 34, wherein the height of contour of the convex surface, or the peak, coincides with the location of slot 32. As such, any pressure that is exerted upon disc 28 in the direction of the tissues is focused and concentrated upon the tissue site immediately covered by pad 34 in the area of slot 32. Increased pressure on a puncture point is known to facilitate desensitization. It is an object of the present invention to focus increased pressure of the cold absorbent surface onto the tissue site and puncture point to facilitate desensitization. When slot 32 is pressed onto a tissue site, the portion of the tissue site that includes the puncture point will push up into slot 32 to form a tissue peak. Blood circulation in the tissues of the tissue peak is reduced. Reduced circulation facilitates tissue cooling and desensitization. The circumferential pressures exerted upon the tissue peak further facilitate desensitization.

It is preferred that the total thickness of disc 28 is minimized, and the disc 28 wall height is low, so as to minimize obstruction to visual and instrument access for the user. As such, the thickness of disc 28 and pad 34 are both minimized.

Needle 26 is shown embedded in a central septum 38. Septum 38 substantially forms a fluid seal about needle 26. Septum 38 extends distally to receiver 30. Receiver 30 and slot 32 are occluded with the distal end of septum 38.

Septum 38 therefore comprises an unblockable fluid blocking means that initially blocks fluid from flowing through needle 26. Septum 38 is unblockable because the blocking function may be undone, such that fluid flow to needle 26 is opened up when needle 26 is sufficiently protruded from pad 34 to a preset depth.

Further, septum 38 prevents the capsule 24 fluid from inadvertently leaking out through slot 32 past needle 26. Septum 38 also seals needle 26 from contamination from external debris.

At least a portion of septum 38 is comprised of an elastomeric material. It is preferred that septum 38 has sufficient stiffness such that substantial pressure is transferable from extension 22 to septum 38, from septum 38 to disc 28, and from disc 28 to the tissue site, so as to increase pressure exertable upon the tissue site. Substantial pressure combined with substantial cold can effectively desensitize a tissue site. However, septum 38 may flex readily.

Holes 40 in the wall of septum 38 comprise a fluid communication from the fluid chamber of capsule 24 to the sidewall of needle 26. When a user presses extension 22 against the proximal wall of capsule 24, the pressure exerted by the proximal wall of capsule 24 upon the fluid contained in the fluid chamber of capsule 24 elastically distends the walls of capsule 24, and thereby substantially pressurizes the contained fluid. As such, capsule 24 comprises a fluid container that contains and pressurizes the fluid to form a pressurized fluid.

However, the lumen of needle 26 is out of fluid communication with the chamber fluid when needle 26 is in an initial position as provided from the manufacturer. Septum 38 seals the pressurized fluid from entering the needle 26 lumen when in the initial position. Fluid pressure will automatically drive the fluid through holes 40 and into needle 26 only after needle 26 is repositioned distally a predetermined distance. It is an object of the present invention to prevent fluid from entering the needle 26 lumen until after the tip of needle 26 has penetrated the tissue site.

The capsule 24 walls are engineered to provide an ideal amount of fluid pressure at the time when the fluid enters the lumen of needle 26. High pressure can cause excessively rapid fluid flow into the tissues and elicit pain. Inadequate fluid pressure may not sufficiently drive an effective amount of the fluid into the tissues. A tissue flow rate of 1 drop per second is preferred into dense tissue.

It is preferred that the fluid in capsule 24 is provided from the manufacturer at a neutral pressure when no manual pressure is exerted upon capsule 24. However, the fluid in capsule 24 may be manufactured pre-pressurized, or even with a negative pressure.

It is preferred that the distal structures of injector 20 function as a needle 26 tissue depth stop. The total thickness of the distal structures of injector 20 is comprised of septum 38, the distal wall of capsule 24, disc 28, and pad 34. The thickness of any of these structures may be specified for the purpose of limiting the needle 26 tissue depth.

As such, when the proximal wall of capsule 24 has collapsed the fluid chamber, and rests firmly against the distal structures of injector 20, then the proximal wall of capsule 24 is limited from further advancement toward the tissue site. When the capsule 24 proximal wall is thus limited from advancement toward the tissue site, the proximal wall and extension 22 will also be limited from advancing needle 26 further toward the tissues, and from advancing the distal tip of needle 26 to a greater tissue depth.

Limiting the tissue depth of needle 26 prevents needle 26 from penetrating beyond a limited zone of desensitization that is provided by the tissue cooling. The tissue depth and breadth of cooling desensitization provided by injector 20 is effective only in a limited area. The tissue depth of effective desensitization can be considered to be about 2 mm. If needle 26 penetrates beyond the 2 mm zone of effective desensitization, the patient is likely to experience pain. Therefore, a capsule 24 depth stop for needle 26 decreases the likelihood of causing pain.

Further, the needle 26 tissue depth is pre-determined so that the tissue site is able to contain the injected fluid. If the tissue can contain the fluid, then the effectiveness of the fluid is maximized, and patient discomfort is minimized. It is preferred that the tip of needle 26 penetrates to a tissue depth of 1.3 mm. Typically, at a 1.3 mm depth, the needle 26 lumen will be sealingly embedded into the tissues, but the tip will not penetrate into sensitive tissue areas beyond the desensitized zone. Further, the initial drops of injected fluid will be deposited into desensitized tissues. Fluids injected into non-desensitized tissues can elicit pain, especially in dense tissue sites. However, other needle 26 penetration depths may be effective. For a needle 26 tip to penetrate to an ideal tissue depth, needle 26 will need to protrude approximately 2 mm from the distal surface of a compressed pad 34.

As such, needle 26 is repositionable to a protruded position by pressure being exerted against injector 20. The protruded position of needle 26 has been repositioned a sufficient distance from the initial non-protruded position such that the fluid blocking means, septum 38, is effectively unblocked to permit automatic fluid flow. However, fluid flow is unblocked only after needle 26 has sealingly penetrated into the tissues.

A socket 42 at the proximal end of capsule 24 is shown connecting to extension 22. Extension 22 has retentive features that secure the connection to socket 42, or extension 22 and socket 42 are unitary, being comprised of a single, homogenous casting. Socket 42 further is reinforced to prevent inadvertent changes in the orientation angle of extension 22 relative to the proximal wall of capsule 24. As such, the likelihood of extension 22 inadvertently rolling the proximal end of capsule 24 laterally when exerting pressure upon capsule 24 is reduced.

It is preferred that the distal end of disc 28, pad 34, and capsule 24 has a shape that somewhat corresponds to the shape, orientation, and angle of the needle 26 bevel. It is further preferred that the leading bevel edge of disc 28 corresponds with the needle 26 bevel tip. As such, a user is able to visually verify the orientation of the needle 26 bevel during tissue puncture.

The distal end of capsule 24 and pad 34 are angled relative to extension 22 so that the needle 26 bevel is able to approach the tissue surface at an angle that is relatively parallel with the tissue surface when pad 34 is in substantially full contact with the tissue surface.

When needle 26 contacts and penetrates the tissue site with the bevel nearly parallel to the tissue surface, the entire bevel and the lumen of needle 26 are entirely enveloped in the tissues at a minimal tissue depth. At the low approach angle, the needle 26 tip does not interfere with the bone just under a shallow tissue site before the lumen is enveloped in the tissues. The enveloped needle 26 lumen forms a fluid seal in the tissues at a shallow tissue depth. As such, the capsule 24 configuration permits needle 26 to sealingly penetrate shallow tissues.

A shallow tissue seal minimizes incident's of needle 26 tip bottoming out on bone in shallow tissue sites before the lumen is sealed in the tissues. If needle 26 were to enter at a more perpendicular angle with respect to the tissue surface, the likelihood is increased that the needle 26 penetration will be prematurely stopped by contact with the bone before the lumen is sealingly embedded into the tissues. If the lumen is not embedded in the tissues, no fluid seal will be formed. Subsequently expressed fluids will not be injected into the tissues, but will instead leak out of the tissues. Leakage increases the likelihood that the patient will experience a problem, such as pain because the tissues are not anesthetized as expected, the injection would have to be repeated, or a needed medicament was not injected as believed. Further, leaked fluid is quickly absorbed by pad 34, which warms pad 34 to negate the cooling desensitization.

In deeper tissue sites, where the bone is covered by more than 2 mm of tissue, the low needle 26 angle negates the need to penetrate beyond the desensitized zone in order to form a tissue seal. On most needles, the bevel itself is approximately 2 mm in length. For needle 26 to penetrate perpendicularly sufficiently for the lumen to be sealingly embedded into the tissues, it is likely that the needle tip would be inserted beyond the depth of reliable desensitization. Because the desensitization caused by the cold from pad 34 decreases rapidly beyond 2 mm in depth into the tissues, the sharp should be restricted to the superficial 2 mm desensitized zone of the tissue site. As such, perpendicular needle 26 penetration angle would increase the likelihood of causing patients pain. Conversely, the lower needle 26 penetration angle permitted by the structure of capsule 24 permits the formation a tissue seal at a shallow tissue depth, decreasing the likelihood of patients experiencing pain.

Chamber 44 is the primary fluid container for capsule 24. It is preferred that a chamber 44 on a first side of septum 38 is in fluid communication with a chamber 44 on a second side of septum 38. As such, chamber 44 comprises a single fluid chamber. However, chamber 44 may be separated by septum 38 into two separate chambers.

The capsule 24 walls about chamber 44 are flexible. When pressure is transferred from extension 22 to the proximal end of capsule 24, the walls of chamber 44 can flex and distend. It is preferred that the chamber 44 walls have substantial resistance to deformation, and therefore do not distend without substantial pressure. The fluid contained in chamber 44 is thereby pressurized to a substantial pressure.

When the fluid in chamber 44 is pressurized to a substantial pressure, then chamber 44 effectively provides substantial backpressure support against the forceful progress of extension 22 and the proximal wall of capsule 24 toward the tissues. Chamber 44 does not collapse readily under pressure. The pressure from extension 22 is thereby substantially transferred to pad 34, and from pad 34 to the tissue site. As such, pad 34 is able to exert substantial pressure on the tissue site. Substantial pressure from a cold pad 34 onto the tissue site enhances the desensitizing effect of the cold.

Needle 26 has at least one port 46. Port 46 comprises a perforation through the lateral wall of needle 26. Port 46 permits fluids to flow into the needle 26 lumen when the port 46 is positioned in fluid communication with a hole 40. Ports 46 are brought into fluid communication with a hole 40 when needle 26 is repositioned distally a given distance from the initial position.

It is preferred that each port 46 of multiple ports 46 is arranged offset from any contralateral port 46, such that opposing ports 46 are not aligned. Once needle 26 has moved a sufficient distance distally, offset ports 46 facilitate uninterrupted fluid communication with holes 40 during continued distal movement of needle 26 past holes 40. However, opposing ports 46 may be aligned on the same axis.

When the proximal wall of capsule 24 exerts pressure upon the fluid in chamber 44, the capsule 24 lateral walls are sufficiently resistant to deformation such that the fluid becomes substantially pressurized. When the pressurized chamber 44 fluid has been brought into fluid communication with the lumen of needle 26 by an alignment of holes 40 and ports 46, then the fluid will automatically flow into the tissue site. The fluid pressure is proportional to the manual pressure exerted upon, and transferred from, extension 22 and socket 42. The manual pressure can be made to be sufficiently high so that pressurized fluid will readily flow, even into dense, fibrous tissue sites such as the oral hard palate, attached gingiva buccal to mandibular molars, and so on.

The user does not need to readjust the finger grip on injector 20, or begin squeezing a particular structure of injector 20, in order to initiate fluid flow from needle 26. User's force vector does not require any change to transition from fluid not flowing to fluid flowing. As such, inadvertent needle 26 movements in the tissues are minimized. Minimizing inadvertent needle 26 movements reduces the chance of eliciting pain for the patient. Further, time is conserved, and the procedure commences is a seamless, stress-free manner for user and patient.

It is preferred that the proximal end of needle 26 is secured to extension 22, such as by embedding the proximal end of needle 26 a given distance into extension 22. Securing needle 26 to extension 22 increases the likelihood that the longitudinal axis of needle 26 and extension 22 will remain in correct alignment during tissue penetration. Securing needle 26 also prevents inadvertent rotation of the bevel tip to an unknown orientation.

Further, securing needle 26 permits it to be withdrawn back into capsule 24 when injector 20 has completed injecting fluid into the tissue site. At the completion of the injection, needle 26 is protruding from the distal end of pad 34. When extension 22 releases pressure against capsule 24, and extension 22 and secured needle 26 move proximally away from the tissue site, then capsule 24 rebounds to its initial non-compressed configuration. When needle 26 is repositioned proximally to its initial position, the distal end of needle 26 is again enclosed within capsule 24.

Alternatively, the proximal end of needle 26 may be embedded in the proximal wall of capsule 24. Needle 26 may have retentive features to provide a purchase for the distal end of needle 26 into the proximal capsule 24 wall. Examples of retentive features include a flared proximal end, perforations, tabs, and so on. However, needle 26 may be non-retentive with capsule 24 or extension 22.

The distal end of extension 22 is shown tapering to a narrow diameter where needle 26 is embedded. As such, when pressure is exerted on extension 22 to compress capsule 24, and extension 22 is forcefully moved toward the tissue site, then the distal end of extension 22 distorts and stretches septum 38 in order to move toward the tissues. The force exerted by the user is sufficient for extension 22 to distort and stretch septum 38, despite septum 38 having substantial resistance to distortion. The narrow end of extension 22 tends to move down the center of septum 38 and stretch the central hole occupied by needle 26. The central stretching of septum 38 facilitates maintaining the alignment of extension 22, needle 26 and septum 38. Septum 38 also tends to compress and flex laterally. Because septum 38 has substantial resistance to distortion, septum 38 transfers substantial pressure from extension 22 to the distal end of capsule 24, to pad 34, and to the tissue site. However, the distal end of extension 22 may be blunt.

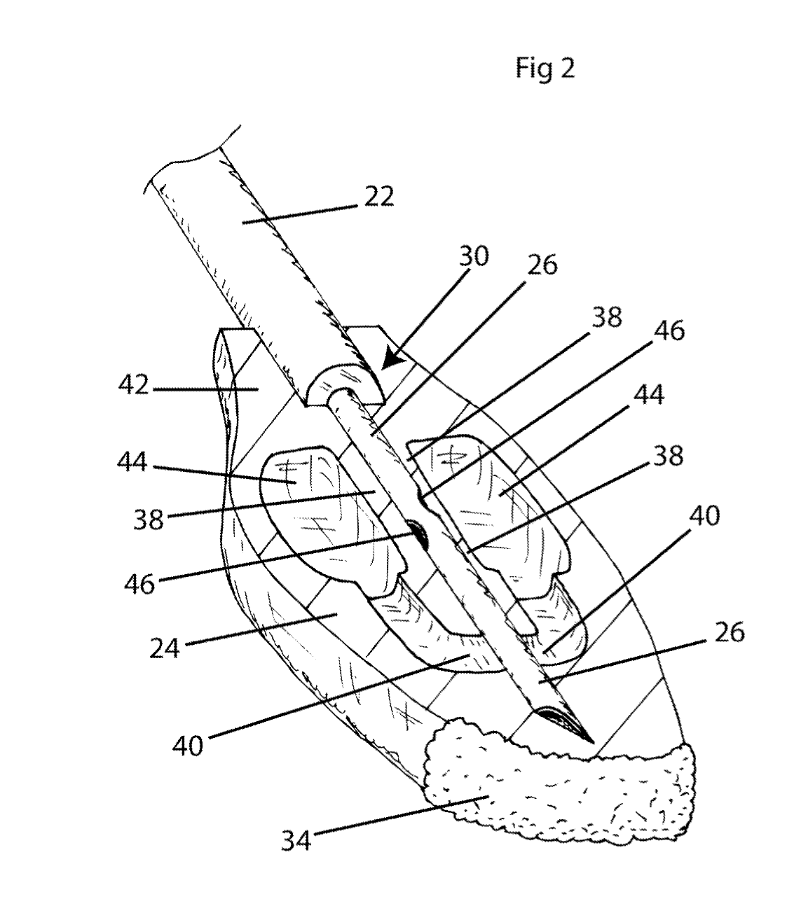

FIG. 2 shows a cross-section of an economical capsule 24 without a disc 28. The configuration of the distal end of capsule 24 is analogous to disc 28, and is capable of exerting pressure against a tissue site.

It is preferred that the shape of the distal end of capsule 24 and pad 34 suggests the shape of the needle 26 bevel so that the user is able to visually verify the orientation of the needle 26 bevel during tissue puncture. Pad 34 covers the distal surface of capsule 24. Pad 34 may be adhered to capsule 24, embedded into the distal outer surface of capsule 24, and so on.

Needle 26 is protrudable from injector 20. The initial position of needle 26 is a nonprotruded position. The area of the distal surface of pad 34 where needle 26 penetrates through in order to protrude beyond pad 34 is considered to comprise a needle 26 protrusion area. Pad 34 substantially encompasses the needle 26 protrusion area.

Needle 26 is shown in an initial position within septum 38. Capsule 24 and septum 38 seal needle 26 from contact with potential contaminants. Needle 26 is further sealed and positioned such that ports 46 are not in fluid communication with capsule 24. As such, fluid will not flow into the needle 26 lumen even when the fluid is pressurized.

The distal end of extension 22 is shown having a blunt end. When extension 22 is pressed toward a tissue site, the blunt end presses against septum 38. Because septum 38 has substantial resistance to distortion, septum 38 transfers substantial pressure from extension 22 to the distal end of capsule 24, to pad 34, and to the tissue site. Pressure applied to the tissue site from a distortion-resistant septum 38 facilitates tissue desensitization.

It is preferred that injector 20 structures function as an automatic needle 26 tissue depth stop. Such depth-stop structures restrict needle 26 to penetrating to a preset depth into the tissue site, and prevent further penetration. The depth-stop structures inhibit further penetration without requiring the user to intentionally alter the insertion pressure or direction in order to limit penetration.

It is preferred that the combined thickness of the proximal capsule 24 wall, the distal capsule 24 wall, and pad 34, functions as a needle 26 tissue depth stop. The thickness of the capsule 24 walls are specified according to the desired needle penetration. When the proximal wall of capsule 24 has collapsed the fluid chamber and firmly contacts the distal structures of injector 20, the proximal wall of capsule 24 is limited from further advancement toward the tissue site, and from pushing the distal tip of needle 26 to an excessive tissue depth. However, other depth stop structures may be utilized.

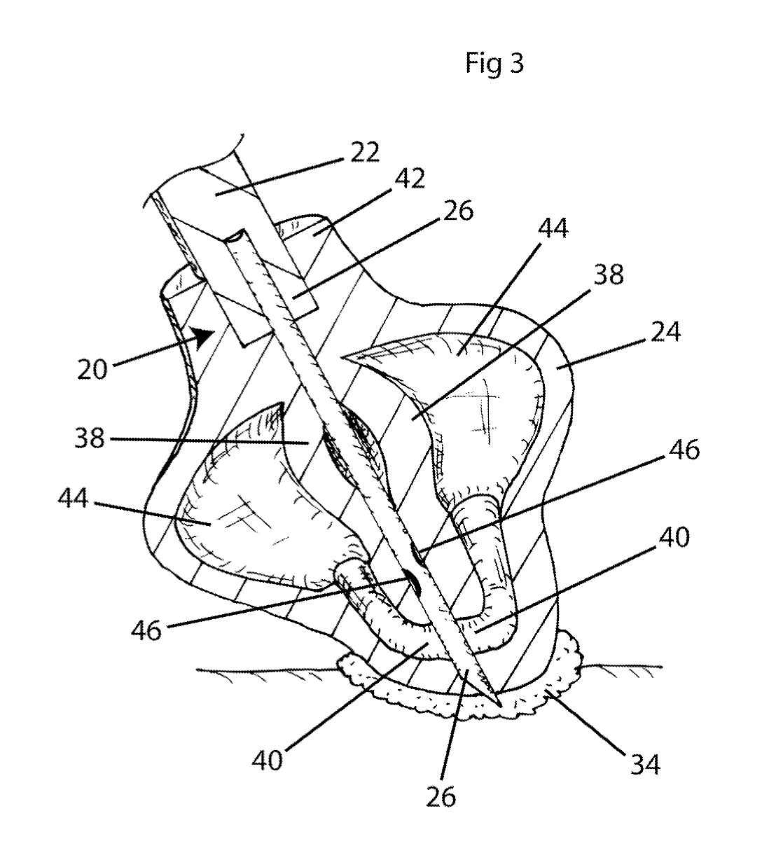

FIG. 3 shows an injector 20 capsule 24 partially compressed by pressure from extension 22 against a tissue site. Socket 42 and the proximal end of capsule 24 are compressed toward chamber 44. The walls about chamber 44 are flexed and partly distended. Extension 22 has partially compressed septum 38. Septum 38 has flexed about needle 26 to allow chamber 44 to partially collapse.

As the proximal wall of capsule 24 is compressed toward the distal wall, and toward the tissue site, needle 26 is repositioned distally toward the tissue site. When capsule 24 is partially collapsed, the fluid contained in chamber 44 is pressurized by the compression and the distension of capsule 24 walls.

As needle 26 continues to be repositioned distally, the sharp distal end of needle 26 penetrates through the distal portion of septum 38, and into pad 34. Needle 26 has not yet been displaced distally a sufficient distance to align holes 40 with ports 46, and thereby bring holes 40 into fluid communication with ports 46. The pressurized fluid has no fluid communication with the needle 26 lumen.

As such, the pressurized fluid contained in chamber 44 remains blocked from flowing through ports 46, and through the lumen of needle 26. With the fluid blocked from flowing through needle 26, the vapocooled pad 34 is not warmed by the fluid. Instead, pad 34 remains cold, and continues to effectively desensitize the tissue site.

As the proximal wall of capsule 24 is further compressed toward the distal than to the degree shown in FIG. 3, and toward the tissue site, the tip of needle 26 begins to penetrate into the tissue site. However, fluid from chamber 44 still does not flow through needle 26 and onto pad 34. Therefore, pad 34 is not warmed by fluid from chamber 44, and remains cold from absorbed vapocoolant. As such, pad 34 continues to effectively desensitize the tissue site.

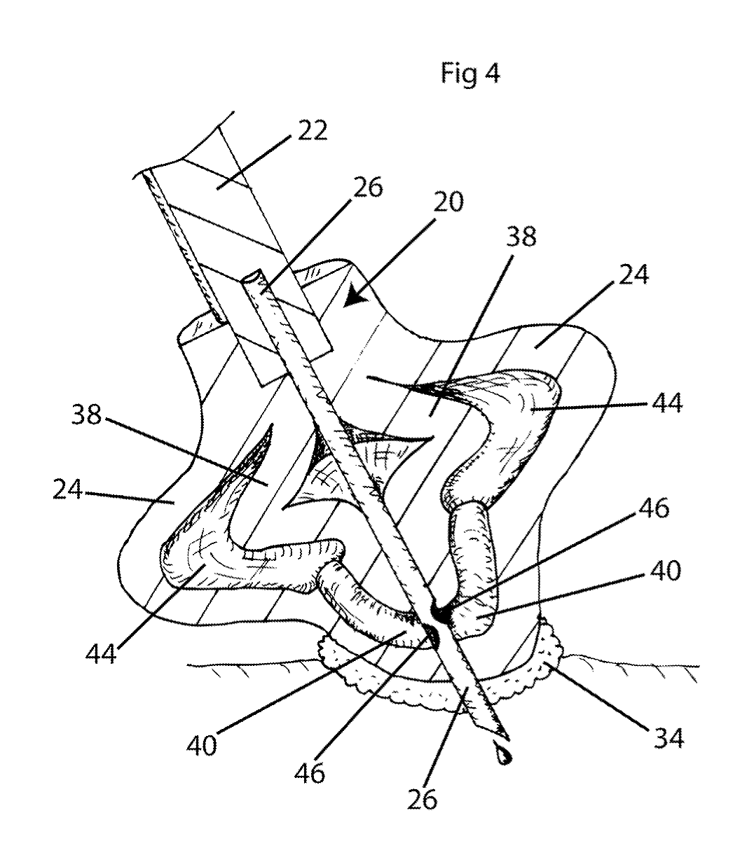

FIG. 4 shows an injector 20 capsule 24 fully compressed by pressure from extension 22 against a tissue site. The proximal end of capsule 24 is compressed toward chamber 44. The walls about chamber 44 are flexed and distended. Extension 22 has penetrated a substantial distance into septum 38. Septum 38 has flexed about needle 26 and extension 22. Chamber 44 is collapsed. When capsule 24 is collapsed, the fluid contained in chamber 44 is pressurized by the compression.

The needle 26 lumen has become entirely embedded in the tissues. When the distal lumen is entirely embedded in the tissues, fluids emitting from the distal lumen are sealed between the tissues and needle 26, and therefore are sealed into the tissues. As such, the needle 26 tip and lumen are sealingly inserted into the tissues.

As the needle 26 tip penetrates the tissue site to the preset depth, ports 46 move into alignment with holes 40, and thereby into fluid communication with holes 40. Pressurized fluid from chamber 44 automatically begins to flow into holes 40, through one or more ports 46 of needle 26, into the lumen of needle 26, and into the tissue site. The fluid is sealingly contained in the tissues, and does not readily leak out of the tissues, and onto cold pad 34. The fluids do not inadvertently warm pad 34. As such, pad 34 continues to chill the tissue site for effective desensitization.

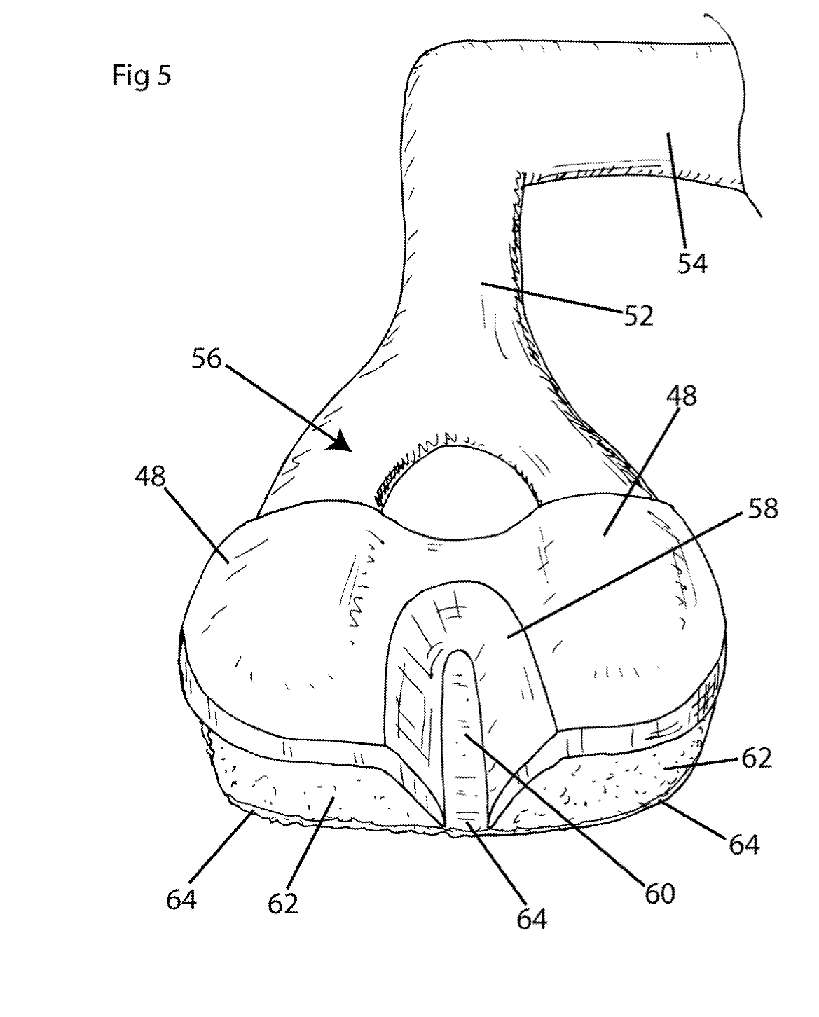

FIG. 5 shows a diskette 48. Diskette 48 is analogous to disc 28. Diskette 48 is intended to comprise a disc 28 having no capsule 24 as an economical embodiment. Diskette 48 may be used to desensitize the tissues prior to an injection, wherein the user provides a separate syringe loaded with medicament in lieu of a pre-charged capsule 24. A user may also use a sharp other than an injection syringe, such as a bone drill, scalpel, and so on.

Diskette 48 is shown having two purchases 50 for securely connecting to the distal end of a forked connector 52. The proximal end of connector 52 is connected to a handle 54 of a hand instrument 56. It is preferred that diskette 48 is detachably connected.

Diskette 48 has a funnel 58 surrounding a central slit 60 for receiving a sharp. Funnel 58 has walls that slope toward slit 60. Such sloping, tapered funnel 58 walls facilitate visualization and instrument access to the puncture point. Further, when a sharp is directed toward slit 60, the sharp tends to be deflected by funnel 58 toward slit 60.

The tissue side of diskette 48 is fluid absorbent. Tissue side absorbency is due to a coolant-absorbent material on the tissue side of diskette 48, felt 62. Felt 62 is comprised of an absorbent material, such as felt, cloth, an array of fibers, and so on. Diskette 48 supports felt 62, such that the user is able to apply substantial pressure from diskette 48 against felt 62, and from felt 62 against the tissue site.

It is preferred that felt 62 is attached to diskette 48 with an adhesive layer interposed between felt 62 and diskette 48. It is preferred that the adhesive layer is associated with felt 62. However, felt 62 may comprise an absorbent slip-on cover placed over either side of diskette 48, an array of absorbent fibers embedded into the surface of diskette 48, and so on. Further, the tissue side of diskette 48 may itself be comprised of an absorbent material, such as a material that is porous, fibrous, corrugated, and so on.

It is preferred that the thickness of felt 62 is somewhat reduced immediately over slit 60. A reduced thickness felt 62 would decrease the overall thickness of diskette 48, further enhancing user visualization and instrument access to the tissue site. A reduced thickness felt 62 would also reduce interference to a sharp from the fibers of felt 62 as a sharp moves through felt 62 and into the tissue site. A reduced thickness of felt 62 over slit 60 may also increase the pressure applied by diskette 48 against the tissue site at the puncture point, which facilitates desensitization. It is preferred that the area of felt 62 reduced thickness does not comprise a sharply delineated area. However, the area may be sharply delineated. The fibers of felt 62 may also be entirely absent in the region of slit 60.

A thin membrane, film 64, may be associated with felt 62, wherein film 64 spans across an area over slit 60 where fibers are sparse or absent. The tissue side of felt 62 is shown having a film 64 spanning across slit 60. Film 64 is used to enhance puncture point cooling, especially where the fibers of felt 62 are sparse or absent. The sharp readily penetrates film 64 immediately prior to penetrating the puncture point. Film 64 may only span across slit 60, it may span across the entire breadth of felt 62, or it may be absent.

It is preferred that film 64 is comprised of an absorbent, fibrous material. However, film 64 may be comprised of a permeable material such that vapocoolant readily penetrates through film 64 and is absorbed into felt 62.

It is preferred that film 64 is comprised of a translucent material. As such, user's visualization of the puncture point is maximized. However, even if film 64 is opaque, the user has an accurate visual representation of the puncture point surface when film 64 is closely adapted onto the puncture point. Film 64 facilitates the user's ability to closely estimate and mentally visualize the surface of puncture point. Film 64 provides the user with substantially unobstructed visual contact with and instrument access to the puncture point.

Alternatively, film 64 may be located on the side of felt 62 that contacts the tissue side of diskette 48, between felt 62 and diskette 48. In this location, film 64 may provide vapocoolant absorbance in the area over the puncture point where the fibers of felt 62 may be sparse. Funnel 58 and a sharp are able to press film 64 into firm contact with the tissue surface.

After the sharp has punctured the puncture point and tissue desensitization is no longer needed, film 64 will permit relocation of felt 62 and diskette 48 away from the sharp while the sharp remains inserted into the tissues. Film 64 is friable and will readily tear against the sharp as felt 62 and diskette 48 are pulled away from the sharp. The sharp will tear film 64 from the point of insertion of the sharp through to the perimeter of film 64, thereby releasing felt 62 and diskette 48 from the sharp. Alternatively, film 64 can be adhered to felt 62 such that film 64 can readily pull free from felt 62. Felt 62 is thereby removable from contact with the tissue site while the sharp remains inserted into the tissues. Such capability to cease felt 62 tissue cooling by removal of felt 62 or diskette 48 from the tissue site prior to removing the sharp protects the tissues against damage or frostbite at effective cooling temperatures.

It is preferred that diskette 48 and funnel 58 provide a convex supporting surface for felt 62, wherein the height of contour of the convex surface, or the peak, coincides with the location of slit 60. As such, any pressure that is exerted upon diskette 48 in the direction of the tissues is focused and concentrated upon the tissue site immediately covered by felt 62 in the area of slit 60. Increased pressure on the tissue site is known to facilitate desensitization.

It is preferred that the total thickness of diskette 48 is minimized, and the diskette 48 wall height is low, so as to minimize obstruction to visual and instrument access for the user. The minimum thickness of diskette 48 is determined by the strength requirements for the material to support felt 62 and maintain connection to connector 52 while under hand pressure against the tissue site.

Similarly, it is preferred that the thickness of felt 62 is minimized. The thickness of felt 62 is determined by the ability of the material to absorb sufficient vapocoolant for effective tissue desensitization. However, felt 62 is not made overly thin, as when felt 62 is too thin, the ability to absorb adequate volume of vapocoolant to cool the tissue site may be compromised. As such, the total thickness of diskette 48 with felt 62 is minimized. A combined thickness of 1 mm, or slightly thicker, provides adequate access for most tissue sites.

It is preferred that slit 60 comprises an elongated hole that is open on one side out to the perimeter of diskette 48. As such, diskette 48 may be removed from about a sharp while the sharp remains inserted into the tissues. Such early removal of diskette 48 from about a sharp facilitates the use of effective coolant temperatures. If felt 62 were to remain in contact with the tissue site substantially beyond the time required for tissue puncture, there is a significant risk of frostbite tissue damage. Therefore the ability to remove diskette 48 from the tissue site prior to removal of the sharp facilitates effective desensitization. As such, the structure of diskette 48 facilitates both safe and effective desensitization of the tissue site. However, slit 60 may comprise a simple round hole in diskette 48, an elongated closed slot, an ovoid hole, and so on.

Further, the perimeter border of slit 60 may comprise a material that is resistant to abrasion, or milling, such as ceramic, metal, certain plastics, and so on. As such, an intraosseous drill may be rotated within slit 60 without abrading or milling the slit 60 perimeter.

Yet further, slit 60 may comprise a perforatable thin area of diskette 48, wherein no hole is present through diskette 48. A needle would perforate the thin slit 60 area of diskette 48, and pass through diskette 48 prior to penetrating the tissue site. Such a perforatable slit 60 enhances tissue desensitization by increasing pressure and cold applied directly to the puncture point.

For a slit 60 configuration that is closed at the perimeter, diskette 48 may be lifted off the tissues prior to removal of a sharp from the tissue site. For example, after a needle is inserted into the tissue site, diskette 48 may be lifted off the tissues by sliding up the needle shaft toward the needle hub. Diskette 48 is held away from the tissues until the sharp is removed from the tissue site to decrease frostbite risk.

It is preferred that slit 60 is in the range of 0.3-6 mm in length, and is 0.3 mm-2 mm in width. More specifically, it is preferred that slit 60 is 1.2 mm in width, and 3 mm in length. A diskette 48 may have two or more slits 60.

It is preferred that diskette 48 is comprised of an injection molded, low heat capacity resin. As such, minimal heat stored within diskette 48 will tend to cause less warming of felt 62 or the tissue site. As such, a low heat capacity diskette 48 facilitates effective tissue site cooling.

It is preferred that the surface of funnel 58 is somewhat absorbent. As such, fluid drops that inadvertently contact funnel 58 may be at least partially absorbed onto the funnel 58 surface. The capability of funnel 58 to absorb inadvertent drops reduces the chance that cold felt 62 will be warmed by fluid drops. Any inadvertent warming of cold felt 62 tends to decrease the desensitizing function of felt 62. The surface of diskette 48 about funnel 58 may also be absorbent.

The absorbent material of funnel 58 or diskette 48 may comprise short perpendicular fibers on the surface of funnel 58 or diskette 48, fibers laying parallel on the surface of funnel 58 or diskette 48, porosity of the material comprising funnel 58 or diskette 48, and so on. It is preferred that any absorbent surface materials of funnel 58 or diskette 48 are substantially thin and low profile so as to avoid obstructing the vision or sharp access to slit 60.

The overall structure of diskette 48 minimizes vision obstruction for the user. When the user has unobstructed vision of the puncture point, user control of the sharp is facilitated, and the risk of causing pain is decreased.

When an effective coolant is applied to a tissue site, the depth of effective and predictable desensitization is limited. In practice, a coolant absorbent surface is able to desensitize to a tissue depth of less than 2 mm. If a needle is inserted beyond this zone of desensitization, the patient will experience a degree of pain. For the coolant desensitization to be effective, the needle must be accurately positionable and controllable within the small zone of desensitization.

Accurate, fine control of the needle tip positioning and movement is facilitated by direct visual contact for the user. The user needs to have direct visual contact with the bevel tip as it initially contacts the tissue surface, and then as it slides into the tissues. Direct vision control prevents the needle tip from wandering, and helps keep it within the small desensitized zone.

Conversely, when the needle is out of direct visual contact, inadvertent needle movements increase the risk of the needle moving outside the small desensitized zone. Inadvertent movements are leveraged by the substantial distance between the needle tip and the user's thumb, commonly 8 inches apart. As such, small hand movements are amplified by the distance to cause significant needle movements. Without continuous direct visual contact of the tissue site to keep the needle in the desensitized zone, the needle is likely to impinge on non-desensitized tissue.

If a few drops of local anesthetic are injected immediately after needle penetration, visual contact also needs to be maintained to confirm that the anesthetic is in fact contained within the tissues, rather than leaking into the mouth. If leakage occurs, the tissues will not be anesthetized according to user expectation, and the patient therefore risks experiencing pain as the procedures commence.

It is preferred that the geometrical configuration of diskette 48 is ovoid. However, diskette 48 may be wedge-shaped, teardrop shaped, round, hexagonal, and so on.

Connector 52 is connected to diskette 48 on the side of diskette 48 that is distal from the user during use. Connector 52 does not substantially obstruct the user's access to diskette 48, nor does any other portion of instrument 56. As such, the user has unobstructed visual and sharps access to the puncture point.

Connector 52 is configured to support diskette 48 in a manner that minimizes obstruction of user visualization and sharps access to diskette 48. Diskette 48 is supported by connector 52 such that slit 60 is aligned with the user's line-of-sight when over a given puncture point. As such, the user has unobstructed visual contact with puncture point when encompassed by slit 60.

Further, connector 52 offsets diskette 48 from handle 54 such that neither handle 54 nor the user's hand tend to block the user's line-of-site visualization or sharps access to the puncture point. Connector 52 is configured so as to offset diskette 48 from handle 54 to further minimize interference with the teeth, other oral structures, the user's fingers, and so on. As such, the user is able to maintain visual contact with the puncture point, and has unobstructed access for instruments and sharps to be used at the puncture point.

The low wall height of diskette 48 permits the needle tip of a syringe to approach the puncture point from a low angle. As such, the beveled needle tip is able to form a fluid seal after insertion to only a shallow depth into the tissue site.

When a needle contacts and penetrates the tissue site with the bevel nearly parallel to the tissue surface, the entire bevel and the lumen of the needle are entirely enveloped in the tissues at a minimal tissue depth. At the low approach angle, the needle tip does not interfere with the bone just under a shallow tissue site before the lumen is enveloped in the tissues. The enveloped needle lumen forms a fluid seal in the tissues at a shallow tissue depth.

A shallow tissue seal minimizes incidents of the needle tip bottoming out on bone in shallow tissue sites before the lumen is sealed in the tissues. If the needle were to enter at a more perpendicular angle, the likelihood is increased that the'needle penetration will be prematurely stopped by contact with the bone before the lumen is sealingly embedded.

In sites where the bone is covered by greater than 2 mm of tissue, the low needle angle negates the need to penetrate beyond the desensitized zone in order to form a tissue seal. The low angle permitted by diskette 48 avoids penetration beyond 2 mm in order to seal the needle lumen with a 2 mm long needle bevel. The lower needle penetration angle permits the formation a tissue seal at a shallow tissue depth, decreasing the likelihood of patients experiencing pain.

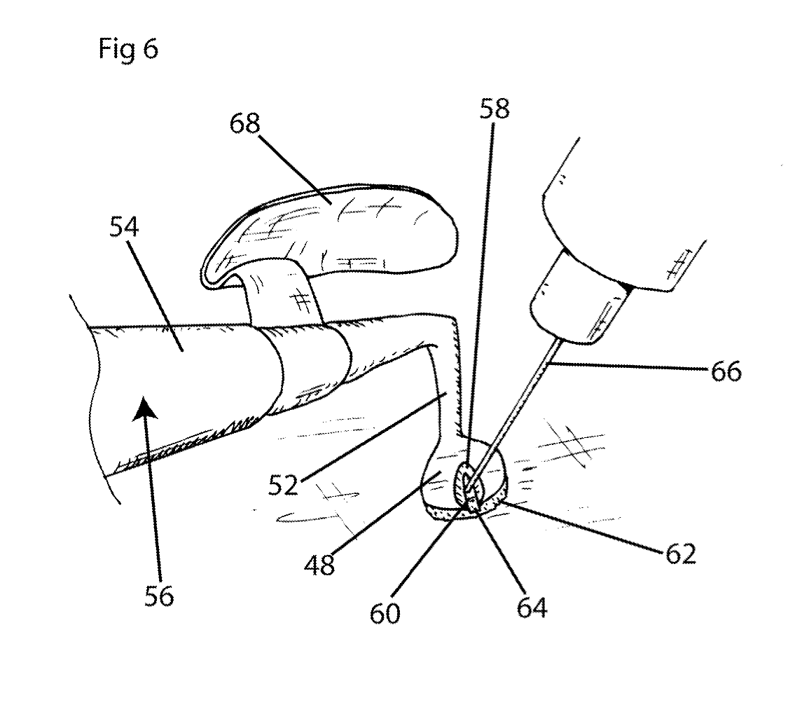

FIG. 6 shows a sharp 66 penetrating a puncture point, and more specifically, a needle sharp 66 that is connected to a syringe, through a diskette 48. The needle sharp 66 is shown inserted through funnel 58, into slit 60, through felt 62 and film 64, and penetrating a puncture point. The user has a substantially unobstructed visual contact with the puncture point, and the needle sharp 66 has unobstructed access to the puncture point. The thin film 64 over the puncture point permits the user to see the exact location of the puncture point surface. Film 64 causes no interference to the insertion of the needle.

A soft tissue retractor 68 is shown extending from handle 54. The retractor is configured to push the lips, cheeks, or tongue, away from the working field to facilitate unobstructed user visualization and instrument access to the puncture point. Retractor 68 is especially useful for deflecting the cheek away from a buccal attached gingiva site for injecting mandibular molars. Alternatively, retractor 68 may extend from connector 52.

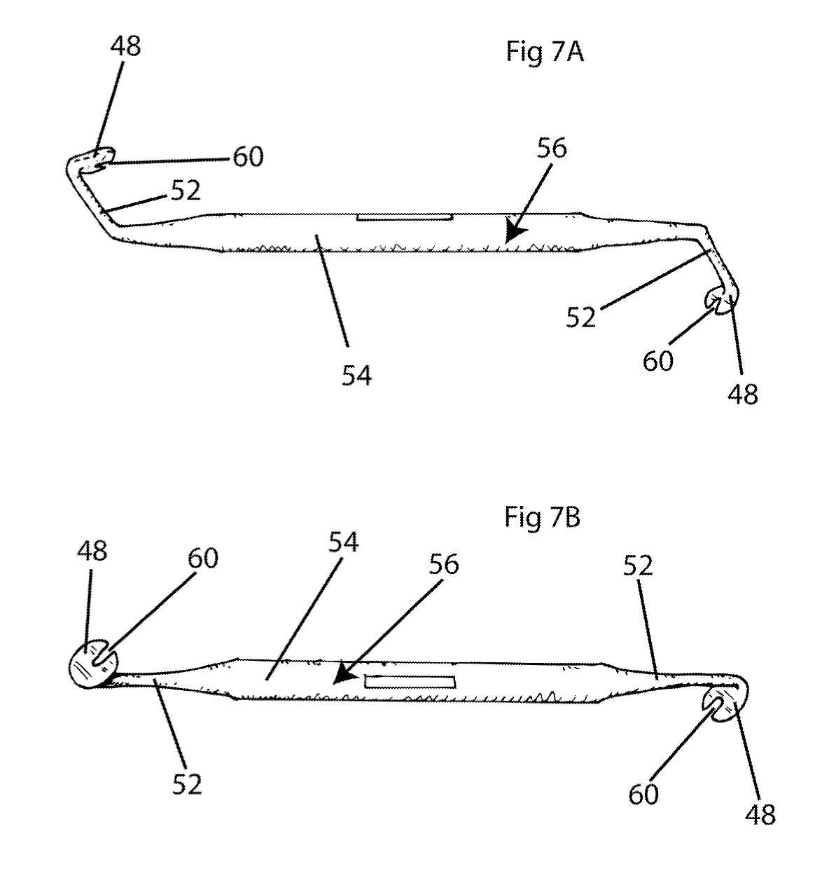

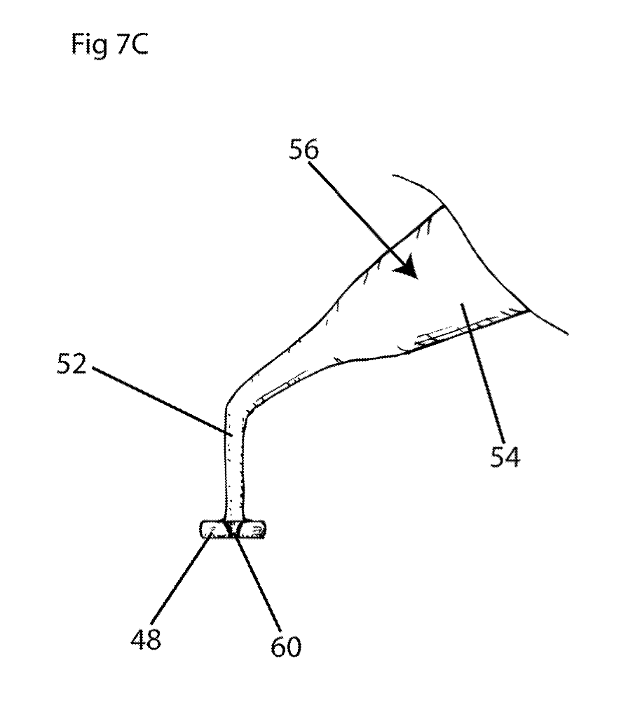

FIGS. 7A-7C are plan views of instrument 56 use to show the orientations of diskette 48 with respect to connector 52 and handle 54. The same plan views would apply for injector 20. The orientation of diskette 48 of instrument 56 is analogous to the orientation of disc 28 of injector 20. Similarly, the orientation of diskette 48 in instrument 56 is analogous to the orientation of the distal end of a capsule 24 with no disc 28. Instrument 56 is shown in FIGS. 7A-7C because the angulations are simpler to illustrate when capsule 24 is absent.

FIG. 7A is an elevation view of instrument 56 showing the orientation of diskette 48 with respect to connector 52 and handle 54 for facilitating user access to the puncture point. Connector 52 is bent 60° away from the long axis of handle 54. The complimentary inside angle of each connector 52 bend is approximately 120°.

The angulation of diskette 48 is 95° with respect to the distal portion of connector 52. Diskette 48 points back toward the user to facilitate user access. This angle positions handle 54 away from the visual line-of-sight and provides the sharp instrument unobstructed access to slit 60 and the puncture point.

FIG. 7B is a top plan view of instrument 56 showing the orientation of diskette 48 with respect to connector 52 and handle 54 for facilitating user access to the puncture point. A line bisecting diskette 48 would be angled 35° away from the axis of handle 54 and connector 52. This angle positions handle 54 away from the visual line-of-sight and provides the sharp instrument unobstructed access to slit 60 and the puncture point. The diskette 48 bisecting line intersects an extended axis of connector 52 and bisects slit 60.

FIG. 7C is an elevation view on instrument 56, from the viewpoint looking toward the distal edge of diskette 48 along the bisecting line. Diskette 48 is oriented so that slit 60 appears in the center. The central axis of each connector 52 is in a vertical orientation. Diskette 48 is oriented perpendicularly with respect to axis of connector 52.

Handle 54 and the proximal portion of connector 52 are shown angling away to the right. Such angulation of diskette 48 facilitates placement of diskette 48 on tissue sites, user visualization, and instrument access. However, other diskette 48 angles with respect to handle 54 and connector 52 may be effective.

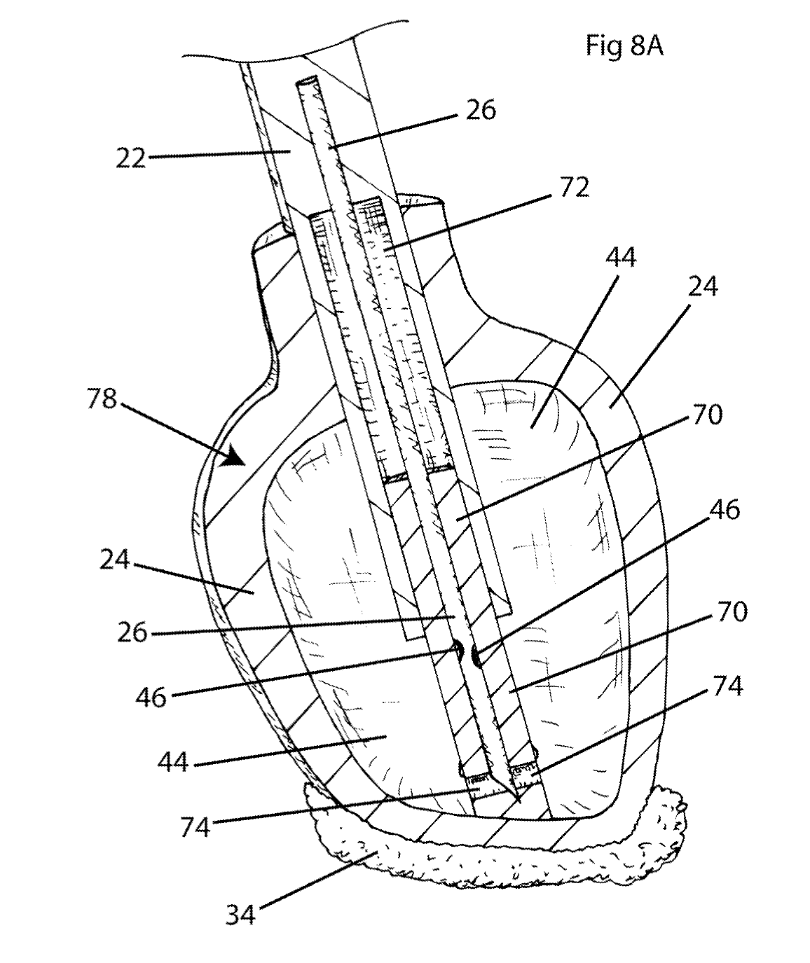

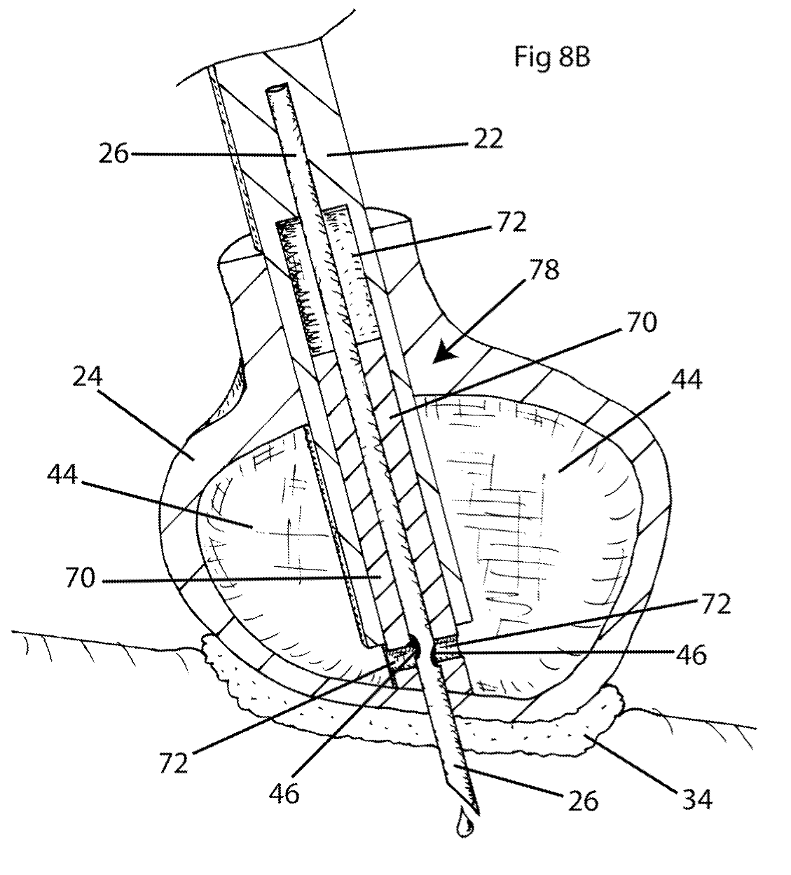

FIGS. 8A and 8B show a capsule 24 having a sleeve 70 over needle 26, substituted for septum 38.

FIG. 8A shows an alternative to septum 38 of capsule 24, sleeve 70. Sleeve 70 comprises a telescoping sleeve surrounding needle 26. Sleeve 70 telescopes into a recess 72 within the distal end of extension 22 when extension 22 is pressed toward a tissue site, and the distal wall of capsule 24 pushes the distal end of sleeve 70 toward extension 22.

Recess 72 is filled with a compressible material that provides resistance to sleeve 70 telescoping into recess 72. The resistance to sleeve 70 telescoping into recess 72 transfers pressure from extension 22 to the distal wall of capsule 24, to pad 34, and to the tissue site. Pressure on the tissue site enhances desensitization.

It is preferred that the compressible material comprises a compressible gel, such as silicone. However, the compressible material may comprise compressible foam, compressible granules, air, and so on.

It is preferred that the distance that sleeve 70 telescopes into recess 72 is limited by a depth stop at a predetermined level. The stop may comprise a diameter restriction, a tab, or ledge, along the inner aspect of recess 72, or on the outer aspect of sleeve 70 near a sleeve hole 74. However, sleeve 70 may be stopped by reaching the limit of compression of the compressible material, by contacting the proximal wall of recess 72, and so on.

Until capsule 24 is compressed, fluid contained within capsule 24 of sleeve injector 76 is blocked from entering ports 46 even when the fluid of chamber 44 is pressurized. Sleeve 70 therefore comprises an unblockable fluid blocking means that initially blocks fluid from flowing through needle 26. Sleeve 70 is unblockable because the blocking function may be undone, so that fluid flow to needle 26 is opened when needle 26 is sufficiently protruded from pad 34. It is preferred that sleeve 70 is cylindrical. However, sleeve 70 may have a rectangular cross-section, or other geometries.

When the user exerts sufficient pressure on extension 22 to compress the compressible material in recess 72 to the point where sleeve 70 contacts the depth stop, then the distal tip of needle 26 penetrates the distal wall of capsule 24, and pad 34. Needle 26 sealingly penetrates into the tissue site, as shown in FIG. 8B. At least one sleeve hole 74 is aligned with ports 46 of needle 26. Pressurized fluid in chamber 44 automatically flows into sleeve hole 74, ports 46, needle 26, and into the tissue site without further manipulation of sleeve injector 76.

As such, needle 26 is repositionable to a protruded position by pressure being exerted against sleeve injector 76. The protruded position of needle 26 has been repositioned a sufficient distance from the initial non-protruded position such that the fluid blocking means, sleeve 70, is effectively unblocked to permit automatic fluid flow. However, fluid flow is unblocked only after needle 26 has sealingly penetrated into the tissues.

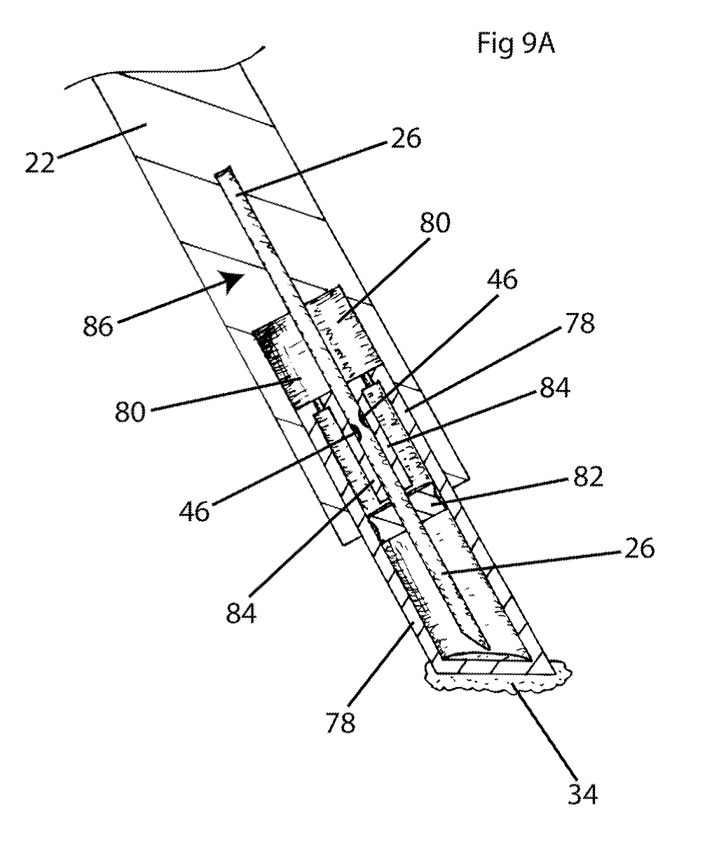

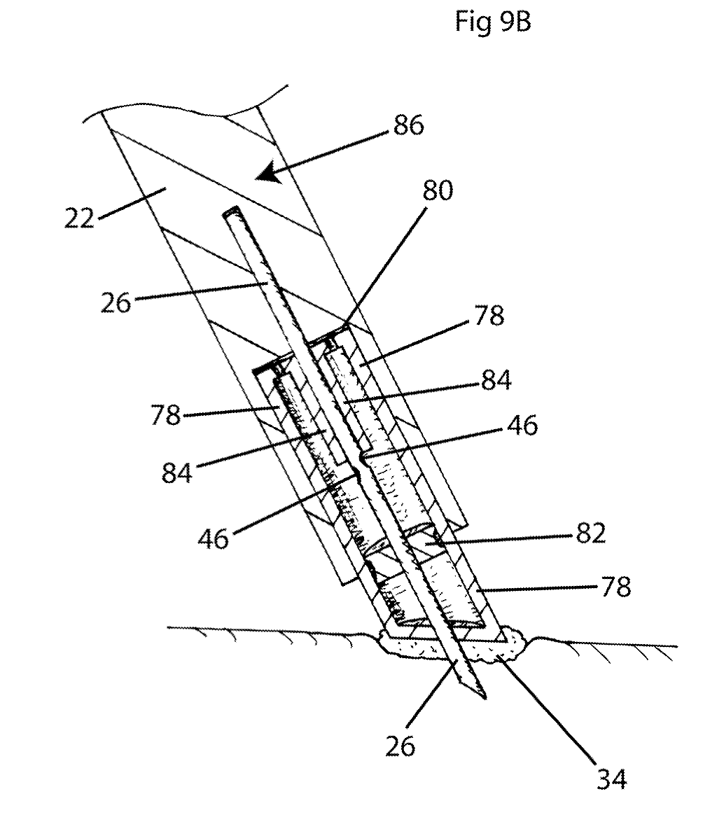

FIGS. 9 A and 9B show an injector having an alternative capsule embodiment utilizing a telescoping piston 78.

FIG. 9A shows telescoping piston 78 in an initial distalized position. Piston 78 sealingly telescopes proximally into a cylinder 80 recessed into the distal end of extension 22 when extension 22 is pressed toward the tissue site.

The external lateral surface of piston 78 forms a fluid seal against the internal surface of cylinder 80, such as with an elastomeric coating, precision fitting, O-rings, or other fluid sealing means.

It preferred that the configuration of the distal end of piston 78 is reminiscent of the bevel of needle 26, and that the bevel of needle 26 is oriented accordingly. As such, a user has a visual representation of the needle 26 bevel orientation. Pad 34 covers the distal end of piston 78.

Needle 26 is protrudable from injector 20. The initial position of needle 26 is a nonprotruded position. The area of the distal surface of pad 34 where needle 26 penetrates through in order to protrude beyond pad 34 is considered to comprise a needle 26 protrusion area. Pad 34 substantially encompasses the needle 26 protrusion area.

Piston 78 is separated into two compartments by a stopper 82, a distal air compartment and a proximal fluid compartment. Stopper 82 substantially blocks fluids in the proximal fluid compartment from occupying the distal air compartment.

The contents of the distal air compartment are compressible. It is preferred that the distal air compartment is filled with air. However, it may be filled with elastomeric closed-cell foam, or other compressible materials. Further stopper 82 may entirely fill the distal air compartment, such as when stopper 82 is comprised of closed-cell foam.

The proximal fluid compartment of piston 78 contains injectable fluid. Cylinder 80 also contains fluid. The proximal end of piston 78 has openings so that the fluid compartment of piston 78 is in fluid communication with cylinder 80. As such, when piston 78 telescopes into cylinder 80, the fluid in cylinder 80 is able to flow through the proximal end openings and into piston 78.

The proximal openings may be restricted to fluid flow so as to provide a shock absorber effect. The restriction of the openings can regulate the time and pressure required to telescope piston 78. The timing and pressure can be selected to coincide with the ideal time and pressure for pad 34 to cool the tissue site for desensitization enhancement.

When fluid moves into piston 78, stopper 82 will compress the air spaces in the distal air compartment to accommodate the space required for the fluid influx. The compressed air spaces and stopper 82 thereby pressurize the contained fluid. As such, piston 78 comprises a fluid container that contains and pressurizes the fluid to form a pressurized fluid.

A flange 84 extends distally from the proximal end of piston 78 to sealingly cover needle 26. Flange 84 blocks pressurized fluid from entering ports 46 of needle 26 until piston 78 has moved a sufficient distance. Fluid is thereby prevented from flowing through needle 26 and inadvertently warming cold pad 34. Flange 84 therefore comprises an unblockable fluid blocking means that initially blocks fluid from flowing through needle 26. Flange 84 is unblockable because the blocking function may be undone, so that fluid flow to needle 26 is opened when needle 26 is sufficiently protruded from pad 34. The piston-type injector, piston injector 86, has the proximal end of needle 26 embedded into extension 22.

FIG. 9B shows piston 78 of piston injector 86 fully telescoped into cylinder 80 as the user presses extension 22 toward the tissue site. Pad 34 is cold from absorption of vapocoolant, and is pressed onto the tissue site.

Fluid formerly in cylinder 80 has flowed into the proximal fluid compartment of piston 78. The air in the distal air compartment is compressed, and is pressing stopper 82 against the fluid. The fluid is thereby pressurized.

Further, the pressurized air presses the distal end of piston 78 against pad 34. As such, cold pad 34 is pressed firmly against the tissue site as an enhancement for desensitization.

As piston 78 telescopes into cylinder 80, the distal end of piston 78 and pad 34 are moved proximally toward extension 22. The distal end of piston 78 is perforated by the tip of needle 26. The tip of needle 26 also pushes through pad 34 as pad 34 moves proximally relative to extension 22.

The tip of needle 26 is exposed beyond pad 34, and penetrates into the tissue site. The patient is aware of the cold of pad 34, but does not experience significant pain from the penetration of needle 26.

The depth of needle 26 penetration is determined by distance that piston 78 telescopes into cylinder 80. The piston 78 telescopic distance is limited by a ledge protruding from the surface of cylinder 80. However, the distance may be limited by the proximal end of piston 78 contacting the proximal end of cylinder 80, by the distal end of extension 22 contacting pad 34 or the tissue surface, and so on.

Flange 84 has moved a sufficient distance proximally along needle 26 to expose ports 46 to the pressurized fluid contained in the proximal fluid compartment. Pressurized fluid automatically flows from the proximal fluid compartment, into needle 26, and into the tissue site.

As such, needle 26 is repositionable to a protruded position by pressure being exerted against piston injector 86. The protruded position of needle 26 has been repositioned a sufficient distance from the initial non-protruded position such that the fluid blocking means, flange 84, is effectively unblocked to permit automatic fluid flow. However, fluid flow is unblocked only after needle 26 has sealingly penetrated into the tissues.

After piston injector 86 is removed from the tissue site, pressurized air in the distal air compartment will expand against, and automatically distalize, the telescoping piston 78. As such, piston 78 rebound to the initial position shown in FIG. 9A, wherein the distal needle 26 tip is covered by piston 78.

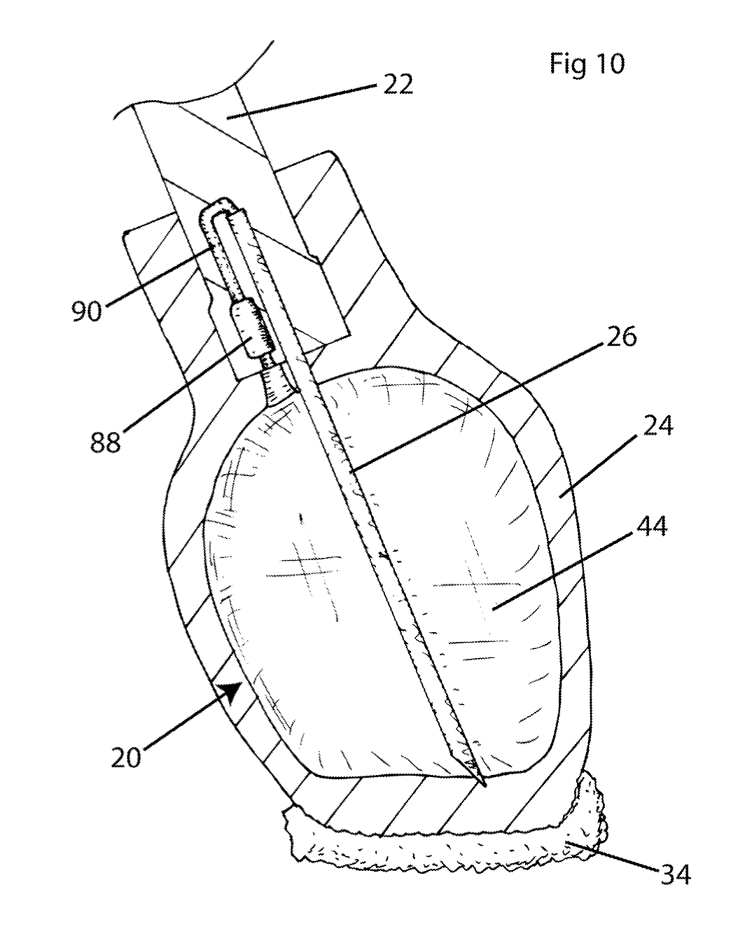

FIG. 10 shows an injector 20 having a pressure-relief valve 88 in fluid communication with chamber 44. Tube 90 connects valve 88 to the needle 26 lumen. Capsule 24 with a valve 88 has the same function as all the other injectors, wherein the automatic flow of pressurized fluid through needle 26 is delayed until after needle 26 is sealingly inserted into the tissues. Pad 34 is on the distal end of capsule 24.

Valve 88 is closed when the fluid in capsule 24 is non-pressurized. Valve 88 is calibrated to open at the fluid pressure that occurs when capsule 24 is compressed by extension 22, and needle 26 is sealingly inserted to a preset tissue depth. After needle 26 is sealingly inserted into the tissues, the fluid in capsule 24 is sufficiently pressurized to open valve 88. When valve 88 opens, the pressurized fluid automatically flows from chamber 44, through valve 88, tube 90, and needle 26, and into the tissues.

Valve 88 may comprise any flow-resistant type valves, including a restricted isthmus tube, a restricted tube, opposing flat elastomeric planes, a ball in an elastomeric socket, a ball and spring, an elastomeric flap, and so on.

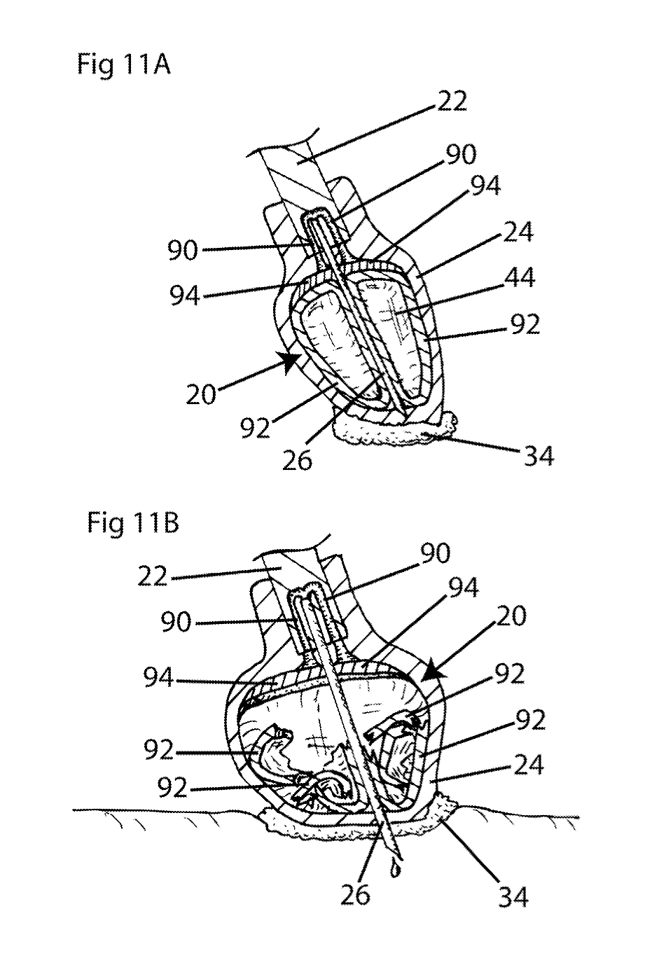

FIGS. 11A and 11B are cross-section views showing an injector 20 having a pressure-rupturable internal shell containing the fluid of chamber 44.

FIG. 11A shows an injector 20 having a rupturable shell 92 contained within chamber 44. Capsule 24 with a shell 92 has the same function as the other injectors, wherein the automatic flow of pressurized fluid through needle 26 is delayed until after needle 26 is sealingly inserted into the tissues. Shell 92 comprises a brittle fluid container.

A porous filter 96 covers the fluid communication opening into tubes 90 to permit fluid flow, but prevent the debris from ruptured shell 92 from blocking fluid flow. Pad 34 is on the distal end of capsule 24.

When capsule 24 is compressed by extension 22, as shown in FIG. 11B, capsule 24 in turn compresses brittle shell 92. When brittle shell 92 is sufficiently compressed, it ruptures and releases the contained fluid. The rupture-resistance of shell 92 is calibrated so that shell 92 ruptures at the degree of compression and stress distortion that occurs after needle 26 has been sealingly inserted to a preset tissue depth.

After needle 26 is sealingly inserted into the tissues, the shell 92 is sufficiently distorted to rupture, and release the fluid into chamber 44. When capsule 24 is compressed sufficiently to rupture shell 92, the fluid released from shell 92 is pressurized from the compression. When shell 92 ruptures, the pressurized fluid automatically flows from chamber 44, through valve 88, tube 90, and needle 26, and into the tissues.

Injector 20 has a porous filter 96 covering the fluid communication opening into tubes 90 to permit fluid flow, but prevent the debris from ruptured shell 92 from blocking fluid flow. Pad 34 is on the distal end of capsule 24.

Other embodiments are also effective for delaying automatic fluid release after needle 26 is sealingly inserted in the tissues. For example, a fluid-filled balloon may be substituted for shell 92. A spike for bursting the balloon is connected to extension 22. After extension 22 has compressed capsule 24 sufficiently to sealingly insert needle 26 into the tissues, the spike impales the balloon and releases the fluid into chamber 44. The fluid is pressurized by the compression, and automatically flows through needle 26 and into the tissues.

From the description above, a number of advantages of the desensitizing instrument become evident:
(a) The instrument concentrates pressure toward the puncture point
(b) The tissue site may be effectively desensitized without substantially obstructing user's visual contact with the puncture point
(c) The tissue site may be effectively desensitized without substantially obstructing sharp access to the puncture point
(d) The instrument facilitates sharps penetrating the puncture point from a convenient and effective angle
(e) The tissue site may be effectively desensitized without substantial tissue damage
(f) Injection fluids are inhibited from inadvertently warming the tissue-coolant surface of the instrument

### Operation

According to another aspect, the invention provides a tissue desensitizing puncture method comprising the steps of: contacting a tissue site with a cold coolant-absorbent surface, inserting a needle through the coolant-absorbent surface to penetrate into a tissue puncture point, wherein fluid is blocked from entering a lumen of the needle until after the needle has penetrated the tissue puncture point, injecting a fluid into the tissue site, removing the coolant-absorbent surface and needle from the tissue site.

The invention provides another tissue desensitizing puncture method comprising the steps of: contacting a tissue site with a cold coolant-absorbent surface, inserting a needle through the coolant-absorbent surface to penetrate into a tissue puncture point, wherein the absorbent surface substantially does not block visual or sharps access to the puncture point, injecting a fluid into the tissue site, removing the coolant-absorbent surface from the tissue site, and removing the needle from the puncture point.

By using the topical press of the invention, it is now possible, surprisingly, to achieve substantial reduction in puncture discomfort within seconds.

The process offers the advantage that the user can now puncture the tissues simply and economically without causing pain.

In a further embodiment of the invention, there are multiple applications of the method for desensitizing tissues with injectors 20, 76, and 86, or instrument 56.

### Example 1

User selects an injector 20 having a disc 28, as shown in FIG. 1, in preparation for pre-anesthetizing the oral hard palate. Injector 20 is connected to a handle 54. The gingiva is a deep tissue site, and has a depth greater than 2 mm.

User applies vapocoolant to felt 62 by spraying Pain Ease (Gebauer) vapocoolant spray for 3 seconds, followed by spraying Endo Ice (Hygienic) for 3 seconds. The vapocoolant evaporates such that pad 34 is made cold, and appears frosty.

Holding handle 54, user observes the configuration of injector 20 to note the orientation of the needle 26 bevel and tip. User orients injector 20 so that the needle 26 bevel will approach the tissue surface from a shallow angle during penetration.

User firmly presses pad 34 onto the surface of the tissue site. The tissue site is made cold by contact with the cold pad 34. Handle 54 firmly presses extension 22 into septum 38, and septum 38 presses firmly against disc 28. The convex tissue side of disc 28 focuses cold pressure from pad 34 against the tissue site at the puncture point. The cold tissue is substantially desensitized for subsequent puncture.

The user further presses capsule 24 against the tissue site. Extension 22 pushes needle 26 toward slot 32. The flexibility of capsule 24 has inadvertently permitted slot 32 to move somewhat laterally, and out of direct alignment with the path of needle 26. The tip of needle 26 contacts the sloping surface of receiver 30, and is deflected into slot 32. Needle 26 proceeds through slot 32, and into pad 34.

As needle 26 is pushed distally toward the tissue site by extension 22, extension 22 also pushes socket 42 and the proximal wall of the capsule 24 distally. Capsule 24 walls flex and distend as the proximal wall pushes distally, but not without substantial resistance. The fluid in chamber 44, a local anesthetic, is pressurized by the manual pressure of extension 22 against the proximal end of capsule 24, and the resistance of the capsule walls to flexing.

Ports 46 are not yet aligned with holes 40. The fluid remains blocked from entering needle 26 while the tip of needle 26 is not embedded into the tissue site. As such, no fluid enters the lumen of needle 26, or is expressed from needle 26. The vapocooled surface of pad 34 is not warmed by undesirable contact with fluids from capsule 24. As such, cold pad 34 continues to effectively desensitize the tissue site.

Extension 22 continues to push needle 26 toward the tissues. The distal end of capsule 24 and pad 34 are angled relative to extension 22 so that the needle 26 bevel is able to approach the tissue surface at an angle that is relatively parallel with the tissue surface when pad 34 is in substantially full contact with the tissue surface.

Needle 26 contacts the tissue site with the bevel nearly parallel to the tissue surface. With further pressure, the tip of needle 26 penetrates through the puncture point of the tissue site. The patient does not experience significant discomfort, but primarily is aware of the cold pad 34.

With the needle 26 bevel oriented nearly parallel to the tissue surface, the entire bevel and the lumen of needle 26 are entirely enveloped in the tissues at a minimal tissue depth. At the low approach angle, the needle 26 lumen is able to form a fluid seal in the tissues at a shallow tissue depth.

As such, the capsule 24 configuration permits needle 26 to form a tissue seal at a shallow tissue depth, thus preventing the need for the user to insert needle 26 to greater depths that may be beyond the desensitized tissue zone.

Extension 22 moves needle 26 distally a sufficient distance to sealingly penetrate the tissues. Ports 46 of needle 26 are located such that they have moved into fluid communication with holes 40. The fluid in chamber 44 is sufficiently pressurized so that it automatically commences to flow through holes 40, through ports 46, into the needle 26 lumen, and is injected into the tissues. User exerts sufficient pressure upon extension 22 so that the fluid is sufficiently pressurized to automatically flow into even dense tissues, such as the attached buccal gingiva opposite mandibular molars.

The user is not required to change the position, grip, or pressure, of the fingers or hand holding injector 20 in order to initiate fluid flow after needle 26 penetrates the tissue site. The fluid flow begins automatically when the needle 26 tip is inserted to a predetermined tissue depth.

Because the user does not need to readjust the grip on injector 20 to inject, inadvertent needle 26 movements within the tissues are minimized. Minimizing inadvertent needle 26 movements reduces the chance of eliciting pain for the patient. The convenient automatic fluid flow facilitates making the injection procedure seamless, rapid, and low-stress.

The tapered distal end of extension 22 penetrates into septum 38, causing septum 38 to distort and spread, despite substantial resistance to distortion. Septum 38 firmly butts against disc 28. The capsule 24 structures work in concert as a depth stop to prevent further movement of extension 22 and needle 26 toward the tissue site. As such, needle 26 is prevented from inserting into the tissues beyond the desensitized zone.

A few drops of deposited local anesthetic fluid are sufficient for pre-anesthetizing the tissue site. Only a few seconds are required for a few drops to be injected into the tissue site. Once sufficient fluid has been deposited, user begins to withdraw extension 22 away from the site.

As the extension 22 pressure that compresses capsule 24 against the tissue site is withdrawn, the resilient capsule 24 walls rebound toward their original shape. As the distal end of capsule 24 rebounds, it pulls itself back over the distal end of needle 26, thereby covering the sharp tip of needle 26.

As injector 20 is withdrawn, the effectively cold pad 34 is removed from the tissue site after only a few seconds of contact. The tissue site immediately begins to warm, and no substantial frostbite damage occurs. Extension 22 is disconnected from handle 54, and extension 22 and capsule 24 are disposed.

After 15 seconds for the tissue site to further pre-anesthetize, user inserts a needle and injects a substantial volume of local anesthetic to profoundly anesthetize a large tissue area about the site for a procedure.

In summary of Example 1, automatic injector 20, containing needle 26 and a fluid, is pressed onto the tissues such that cold pad 34 is desensitizingly pressed onto the tissue surface. The pressure thereby pressurizes the fluid to form a pressurized fluid. The pressurized fluid is initially blocked from flowing through needle 26 by septum 38. The pressure protrudes needle 26 a sufficient distance from injector 20 such that needle 26 sealingly penetrates the tissues. When needle 26 reaches a sufficient depth into the tissue for sealing penetration, then the pressurized fluid is unblocked by septum 38 by the alignment of holes 40. The pressurized fluid then automatically flows through needle 26 and into the tissues.

### Example 2

User selects an injector 20 having no disc 28, as shown in FIG. 2, in preparation for pre-anesthetizing a tissue site on the buccal surface of mandibular molars. Injector 20 is not connected to a handle 54. Injector 20 comprises an extension 22, a capsule 24, and a needle 26 preloaded with local anesthetic. The gingiva is a shallow tissue site, and has a depth of 2 mm.

User applies vapocoolant to pad 34 by spraying Pain Ease (Gebauer) vapocoolant spray for 3 seconds, followed by spraying Endo Ice (Hygienic) for 3 seconds. The vapocoolant evaporates such that pad 34 is made cold, and appears frosty.

Holding extension 22, user observes the configuration of injector 20 to note the orientation of the needle 26 bevel and tip. User orients injector 20 so that the needle 26 bevel will approach the tissue surface from a shallow angle during penetration.

User firmly presses pad 34 onto the surface of the tissue site. The tissue site is made cold by contact with the cold pad 34. Extension 22 is firmly pressed into septum 38, and septum 38 presses firmly against the distal wall of capsule 24. The convex tissue side of the distal wall focuses cold pressure from pad 34 against the tissue site at the puncture point. The cold tissue is substantially desensitized for subsequent puncture.

The user further presses capsule 24 against the tissue site. Extension 22 pushes needle 26 through the distal wall of capsule 24, and into pad 34, as shown in FIG. 3.

As needle 26 is pushed distally toward the tissue site by extension 22, extension 22 also pushes socket 42 and the proximal wall of the capsule 24 distally. Capsule 24 walls flex and distend as the proximal wall pushes distally, but not without substantial resistance. The fluid in chamber 44, a local anesthetic, is pressurized by the manual pressure of extension 22 against the proximal end of capsule 24, and the resistance of the capsule walls to flexing.

Ports 46 are not yet aligned with holes 40. The fluid remains blocked from entering needle 26 while the tip of needle 26 is not embedded into the tissue site. As such, no fluid enters the lumen of needle 26, or is expressed from needle 26. The vapocooled surface of pad 34 is not warmed by undesirable contact with fluids from capsule 24. As such, cold pad 34 continues to effectively desensitize the tissue site.

Extension 22 continues to push needle 26 toward the tissues. The distal end of capsule 24 and pad 34 are angled relative to extension 22 so that the needle 26 bevel is able to approach the tissue surface at an angle that is relatively parallel with the tissue surface when pad 34 is in substantially full contact with the tissue surface.

Needle 26 contacts the tissue site with the bevel nearly parallel to the tissue surface. With further pressure, the tip of needle 26 penetrates through the puncture point of the tissue site. The patient does not experience significant discomfort, but primarily is aware of the cold pad 34.

With the needle 26 bevel oriented nearly parallel to the tissue surface, the entire bevel and the lumen of needle 26 are entirely enveloped in the tissues at a minimal tissue depth. At the low approach angle, the needle 26 lumen is able to form a fluid seal in the tissues at a shallow tissue depth.

The shallow tissue seal permits fluid injection into the shallow site. The needle 26 tip does not penetrate to a depth where the tip would interfere with the shallow bone before the lumen is enveloped in the tissues.

Extension 22 moves needle 26 distally a sufficient distance to sealingly penetrate the tissues. Ports 46 of needle 26 are located such that they have moved into fluid communication with holes 40, as shown in FIG. 4. The fluid in chamber 44 is sufficiently pressurized so that it automatically commences to flow through holes 40, through ports 46, into the needle 26 lumen, and is injected into the tissues. User exerts sufficient pressure upon extension 22 so that the fluid is sufficiently pressurized to automatically flow into even dense tissues, such as the attached buccal gingiva opposite mandibular molars.

The user is not required to change the position, grip, or pressure, of the fingers or hand holding injector 20 in order to initiate fluid flow after needle 26 penetrates the tissue site. The fluid flow begins automatically when the needle 26 tip is inserted to a predetermined tissue depth.

Because the user does not need to readjust the grip on injector 20 to inject, inadvertent needle 26 movements within the tissues are minimized. Minimizing inadvertent needle 26 movements reduces the chance of eliciting pain for the patient. The convenient automatic fluid flow facilitates making the injection procedure seamless, rapid, and low-stress.

Extension 22, the capsule 24 proximal wall, and septum 38, firmly butt against the distal capsule 24 wall. The wall is thickened to include the area of the holes 40. The capsule 24 structures work in concert to prevent further movement of extension 22 and needle 26 toward the tissue site. As such, needle 26 is prevented from inserting into the tissues beyond the desensitized zone.

A few drops of deposited local anesthetic fluid are sufficient for pre-anesthetizing the tissue site. Only a few seconds are required for a few drops to be injected into the tissue site. Once sufficient fluid has been deposited, user begins to withdraw extension 22 away from the site.

As the extension 22 pressure that compresses capsule 24 against the tissue site is withdrawn, the resilient capsule 24 walls rebound toward their original shape. As the distal end of capsule 24 rebounds, it pulls itself back over the distal end of needle 26, thereby covering the sharp tip of needle 26, as shown in FIG. 1.

As injector 20 is withdrawn, the effectively cold pad 34 is removed from the tissue site after only a few seconds of contact. The tissue site immediately begins to warm, and no substantial frostbite damage occurs. Injector 20 is disposed.

After 15 seconds for the tissue site to further pre-anesthetize, user bores through the tissue site and the cortical bone with an intraosseous drill bit. The drill bit penetrates into the medullary bone. Once the hole is bored, a local anesthetic is injected into the medullary bone. After approximately 15 seconds after the anesthetic has been injected into the bone, the mandibular molars are sufficiently anesthetized to commence a dental procedure on the molar teeth.

In summary of Example 2, automatic injector 20, containing needle 26 and a fluid, is pressed onto the tissues such that cold pad 34 is desensitizingly pressed onto the tissue surface. The pressure thereby pressurizes the fluid to form a pressurized fluid. The pressurized fluid is initially blocked from flowing through needle 26 by septum 38. The pressure protrudes needle 26 a sufficient distance from injector 20 such that needle 26 sealingly penetrates the tissues. When needle 26 reaches a sufficient depth into the tissue for sealing penetration, then the pressurized fluid is unblocked by septum 38 by the alignment of holes 40. The pressurized fluid then automatically flows through needle 26 and into the tissues.

### Example 3

In preparation for an intraosseous injection, purchases 50 of a diskette 48 are connected to connector 52 of an instrument 56, as shown in FIG. 5 and FIGS. 7A-C. User applies vapocoolant to felt 62 and a film 64 on diskette 48. The vapocoolant evaporates such that felt 62 and film 64 are made cold, and appear frosty.

Instrument 56 is inserted into the mouth. Diskette 48 is oriented over a tissue site such that slit 60 is aligned to face toward the user. Retractor 68 deflects the lips and cheek away from the buccal surfaces of the mandibular molars, as shown in FIG. 6. User is afforded a direct and unobstructed view of the tissue site.

Felt 62 and film 64 are pressed onto a shallow, 2 mm depth, buccal tissue site. The tissue site is made cold by contact with the cold felt 62 and film 64. The cold tissue is substantially desensitized for subsequent puncture. The user further presses diskette 48 against the tissue site. The tissue side of funnel 58 focuses pressure onto felt 62, and felt 62 presses firmly onto the tissue site. The focusing of pressure enhances the desensitization from the cold from felt 62 onto the tissue site.

Film 64 is closely adapted to the tissue surface like a second skin. User is able to see the puncture point through the transparent film 64, and maintains continuous visual contact with the puncture point as felt 62 desensitizes the tissue site. Film 64 causes no substantial interference to user's vision of the puncture point.

User approaches the puncture point with a sharp 66 comprising a beveled needle attached to a syringe. No part of instrument 56 causes any substantial obstruction to the approach. Without obstruction, user is able to rapidly approach and position sharp 66 from an ideal angle.

Upon approach to slit 60, the needle of sharp 66 inadvertently contacts the sloping surface of funnel 58. Funnel 58 redirects sharp 66 toward slit 60.

Despite user's attempt to prevent fluid leakage from the sharp 66 needle, a drop of room-temperature fluid from the syringe expresses from the sharp 66 needle onto the funnel 58 surface. Funnel 58 absorbency fibers substantially absorb the relatively warm fluid, and prevent it from becoming absorbed by cold felt 62. Funnel 58 absorbency minimizes warming of felt 62 by inadvertent drops of fluid from the sharp 66 needle. As such, funnel 58 absorbency facilitates effective desensitization of the tissue site.

The sharp 66 needle proceeds through slit 60, and into felt 62. The sharp 66 bevel is able to approach the tissue surface at an ideally low angle so that the bevel is nearly parallel with the tissue surface. With further pressure, the tip of sharp 66 penetrates through film 64 and into the tissue puncture point, as shown in FIG. 6. The patient does not experience significant discomfort, and primarily senses only the cold from felt 62.

The diskette 48 configuration permits needle sharp 66 to sealingly penetrate at a shallow tissue depth. With the sharp 66 bevel oriented nearly parallel to the tissue surface, the entire bevel and the lumen of sharp 66 are entirely enveloped in the tissues at a shallow tissue depth of approximately 1 mm. At the low approach angle, the sharp 66 tip does not interfere with the bone 2 mm under the shallow tissue site before the lumen is enveloped in the tissues. The sharp 66 needle lumen is able to form a fluid seal with the tissues at the shallow tissue depth of 1 mm. The shallow tissue seal avoids bottoming the sharp 66 needle tip on the bone before the lumen is sealingly embedded.

User pressurizes the fluid in the syringe, a local anesthetic, so that a few drops of fluid begins to flow through the needle sharp 66 lumen, and is injected into the tissues. The tissues sealingly contain the injected fluid, and prevent the warm fluid from leaking onto, and warming, the cold felt 62 surface. Therefore, the tissue site remains adequately desensitized. Further, the local anesthetic fluid quickly anesthetizes the tissue site.

Several seconds after the fluid injection commences, user begins to withdraw diskette 48 away from the tissue site. User lifts diskette 48 off the tissue, thereby removing cold felt 62 with film 64 from the tissue site. Such removal of diskette 48 from the tissues a short time after penetration prevents frostbite damage at the effectively cold desensitization temperatures.

For convenience, user also moves diskette 48 laterally away from sharp 66, so that sharp 66 tears through film 64, slides out through slit 60, slides past the perimeter of diskette 48, and becomes free of diskette 48. User removes diskette 48 from the field of operation.

Needle sharp 66 remains in the tissues until the fluid injection is complete. After the injection is complete, user withdraws sharp 66 from the tissue site.

After approximately 20 seconds for the deposited local anesthetic to anesthetize the tissue site, an intraosseous drill, X-Tip (Dentsply), is used to perforate through the tissue site gingiva, through the cortical plate bone, and into the medullary bone, to form a bony hole. Additional anesthetic is deposited through the bony hole to anesthetize the adjacent teeth and gingiva for subsequent procedures.

### Example 4

In preparation for an intraosseous injection of the mandibular molars, a user selects an instrument 56 having a diskette 48 with a felt 62 and film 64, as shown in FIG. 5 and FIGS. 7A-7C. Vapocoolant is sprayed onto felt 62 and film 64. Diskette 48 is oriented over a tissue site such that slit 60 is aligned to face toward the user. Retractor 68 deflects the lips and cheek away from the buccal surfaces of the mandibular molars to improve site access. After a few seconds of evaporation, frosty felt 62 and film 64 are firmly pressed against a tissue site.

The cold effectively desensitizes the tissue site to a depth of approximately 2 mm. The shallow tissue site is 2 mm in depth over the cortical bone. The tissue side of funnel 58 focuses pressure onto felt 62, and felt 62 presses firmly onto the tissue site. The focusing of pressure enhances the desensitization from the cold from felt 62 onto the tissue site. The tissue site is effectively desensitized through to the depth of the cortical bone.

The thin, transparent film 64 clings to the tissue site and visually reveals the puncture point surrounded by slit 60 to the user. Neither film 64 nor felt 62 substantially obstructs user visual access or instrument access.

An intraosseous drill sharp 66 is inserted into slit 60 without substantial obstruction to the user from either handle 54, connector 52, diskette 48, felt 62, film 64, or user's fingers. Drill sharp 66 is touched to the insertion side of film 64 directly over the puncture point. The user has an unobstructed view of drill sharp 66 touching the surface of the puncture point, and an unobstructed pathway for drill sharp 66 to access the puncture point. Drill sharp 66 is rotated for a few seconds to bore a hole through film 64, through the tissues, through the cortical bone, and into the medullary bone in a few seconds to form a bony hole. During the boring with drill sharp 66, the patient feels the cold from felt 62 but does not experience pain from boring drill sharp 66 due to effective desensitization. The bone itself typically does not provide a painful sensation when perforated by a small drill. Drill sharp 54 is removed from the bony hole and tissue site. Diskette 48 is removed from the tissue site.

An intraosseous needle, or a sleeve for receiving an intraosseous needle, is inserted into the bony hole. Local anesthetic is deposited through the bony hole and into the medullary bone. The teeth and tissues adjacent to the bony hole are profoundly anesthetized within a few seconds. User is able to perform a procedure on the teeth or gingival tissues without the patient experiencing pain.

### Example 5

A vapocoolant is applied to pad 34 of a sleeve injector 76, as shown in FIG. 8A. User observes the configuration of capsule 24 to note the orientation of the needle 26 bevel and tip. User orients sleeve injector 76 so that the needle 26 bevel will approach the tissue surface from a shallow angle during penetration.

Pad 34 is pressed firmly onto the tissue site. The distal wall of capsule 24 pushes the distal end of sleeve 70 proximally toward extension 22 such that sleeve 70 telescopes into recess 72. The compressible material contained in recess 72 compresses to permit sleeve 70 to telescope into recess 72. The chamber 44 fluid becomes pressurized as the walls of compressed capsule 24 distend. Sleeve 70 blocks the pressurized fluid from entering ports 46 of needle 26.

As user further presses capsule 24 against the tissue site, extension 22 pushes needle 26 through the distal wall of capsule 24, and into pad 34. Sleeve 70 slides proximally relative to needle 26. However, sleeve 70 continues to block the fluid, so that pad 34 remains free of fluid from chamber 44.

The compressible material provides increasing compression resistance to the telescoping sleeve 70 as the telescoping progresses. The pressure is transferred to pad 34, and the tissue site is further desensitized by the cold pressure.

Needle 26 contacts the tissue site with the bevel nearly parallel to the tissue surface, and sealingly penetrates to a shallow depth into the tissue site. The patient does not experience significant discomfort, but primarily is aware of the cold pad 34.

The depth stop interferes with further telescoping of sleeve 70 into recess 72, thereby limiting the tissue penetration depth of needle 26 for patient comfort.

Ports 46 of needle 26 have moved into fluid communication with sleeve holes 62, as shown in FIG. 8B. The fluid in chamber 44 is sufficiently pressurized so that it automatically commences to flow through sleeve holes 62, through ports 46, into the needle 26 lumen, and is injected into the tissues. The user does not manipulate sleeve injector 76 to initiate fluid flow.

After a few seconds, a few drops have been injected into the tissue site, and user withdraws extension 22 from the tissues. Resilient capsule 24 and the compressible material in recess 72 rebound the distal wall of capsule 24 back over the tip of needle 26, thereby covering the sharp tip of needle 26, as shown in FIG. 8A. The tissue site immediately begins to warm, and no substantial frostbite damage occurs. Sleeve injector 76 is disposed.

In summary of Example 5, automatic sleeve injector 76, containing needle 26 and a fluid, is pressed onto the tissues such that cold pad 34 is desensitizingly pressed onto the tissue surface. The pressure thereby pressurizes the fluid to form a pressurized fluid. The pressurized fluid is initially blocked from flowing through needle 26 by sleeve 70. The pressure protrudes needle 26 a sufficient distance from sleeve injector 76 such that needle 26 sealingly penetrates the tissues. When needle 26 reaches a sufficient depth into the tissue for sealing penetration, then the pressurized fluid is unblocked by repositioning sleeve 70 proximally along needle 26 so that sleeve holes 74 are in fluid communication with ports 46. The pressurized fluid then automatically flows through needle 26 and into the tissues.

### Example 6

User selects a piston injector 86, as shown in FIG. 9A. User applies vapocoolant to pad 34. Observing the configuration of piston 78, user orients the piston injector 86 so that needle 26 bevel is mostly parallel with the tissue surface as pad 34 contacts the tissue.

As extension 22 is firmly pressed toward the tissues, pad 34 and the distal end of piston 78 begin to telescopically move the body of piston 78 into cylinder 80. With significant resistance, fluid moves through holes in the proximal end of piston 78, permitting piston 78 to move into cylinder 80. Significant pressure is transferred to pad 34, and to the tissue site, enhancing desensitization.

The air in the distal air compartment of piston 78 compresses, and pushes against stopper 82. Stopper 82 pressurizes the fluid in the proximal fluid compartment. Flange 84 has not yet moved sufficiently to expose ports 46 of needle 26, so the fluid remains blocked from entering needle 26. Pad 34 remains unwarmed by inadvertent fluid contact.

The user further presses piston 78 against the tissue site, and piston 78 telescopes further into cylinder 55. Needle 26 pushes through the distal wall, into pad 34, and penetrates to a shallow depth into the tissue site, as shown in FIG. 9B. The needle lumen is sealingly embedded into the tissues at a shallow depth due to the low needle bevel approach angle wherein the bevel was somewhat parallel to the tissue surface. The patient does not experience significant discomfort, but is only aware of the cold pad 34. The proximal wall of piston 78 contacts the stop in the proximal portion of cylinder 80. Flange 84 has moved a sufficient distance to expose ports 46 of needle 26. The pressurized fluid in the proximal fluid compartment automatically moves through ports 46, into the needle 26 lumen, and is injected into the tissues. User does not have to manipulate piston injector 86 to initiate fluid flow.

After a few drops of fluid are deposited, user begins to withdraw extension 22 away from the site. The effectively cold pad 34 is removed from the tissue site after only a few seconds of contact, so risk of frostbite is minimized.

The distal air in piston 78 rebounds the distal wall back over the tip of needle 26, thereby covering the sharp tip of needle 26, as shown in FIG. 9A. The tissue site immediately begins to warm, and no substantial frostbite damage occurs. Piston injector 86 is disposed, and user proceeds with further treatment.

In summary of Example 6, automatic piston injector 86, containing needle 26 and a fluid, is pressed onto the tissues such that cold pad 34 is desensitizingly pressed onto the tissue surface. The pressure thereby pressurizes the fluid to form a pressurized fluid. The pressurized fluid is initially blocked from flowing through needle 26 by the distal positioning of flange 84. The pressure protrudes needle 26 a sufficient distance from piston injector 86 such that needle 26 sealingly penetrates the tissues. When needle 26 reaches a sufficient depth into the tissue for sealing penetration, then the pressurized fluid is unblocked by repositioning flange 84 proximally along needle 26 to expose ports 46. The pressurized fluid then automatically flows through needle 26 and into the tissues.

### SUMMARY

In summary, lower coolant temperatures are more effective for desensitizing tissue sites. The injectors and instrument 56 permit the use of lower cooling temperatures than are safe utilizing other methods at least in part because they minimize the tissue cooling time required for effective desensitization and treatment.

Tissue cooling time is minimized because pad 34 and felt 62 substantially surround the puncture point. A substantially surrounded puncture point cools faster than a site cooled from only one side. This decreases the cooling time required to effectively desensitize the tissue prior to puncture.

Tissue cooling time is also minimized because instrument 56 provides an unobstructed line-of-sight to the puncture point, and unobstructed access for sharps to access the puncture point. After instrument 56 begins to cool the tissues, the user is able to position sharp 66 in preparation for puncture in less time than when obstructions are present. This reduces the cooling time required between desensitization and the tissue puncture. The configuration of the injectors also minimizes tissue site obstruction and injection time.

Unobstructed access also reduces cooling time required for the user to complete the puncture and manipulation of sharp 66 in the tissue until desensitization is no longer needed. For example, open access reduces the time needed to comfortably advance a local anesthetic needle sharp 66 into the tissue, and to comfortably deposit sufficient anesthetic. As such, needle sharp 66 may comfortably remain in the tissues to complete an injection after instrument 56 is removed.

Tissue cooling time is also minimized because the injectors and instrument 56 coolants are applied only to small target tissue site. When only a small site is cooled, tissue rewarming occurs rapidly after removal of the injectors or instrument 56 because of the small site diameter requiring heat conduction.

Tissue cooling time is also minimized because diskette 48 and felt 62 are removable from the tissue before sharp 66 is ready to be removed from the tissue. Removal of diskette 48 and felt 62 prior to removal of sharp 66 reduces overall tissue cooling time.

Tissue cooling time is minimized for the injectors because the single-motion efficiency of the automatic injection permits abbreviated cooling times. Such abbreviated cooling times are comparable to those achieved by removing instrument 56 from the site prior to completing a sharp 66 injection.

Tissue cooling time is also minimized because only a minimal amount of coolant is transferred to the tissue from pad 34 or felt 62. The transferred amount is minimized primarily because the coolant is allowed to crystallize on the absorbent surface prior to contacting the tissue site. As a result, the tissue rewarms quickly after removal of pad 34 or felt 62.

The above factors substantially minimize cooling time, thereby permitting lower and more effective cooling temperatures than are achievable utilizing other methods.

An alternative pad 34 or felt 62 coolant comprises water absorbed into the absorbent surface and pre-frozen in a freezer. Another alternative coolant method comprises a pad 34 or felt 62 presoaked with vapocoolant, and sealed. When such a seal about pad 34 or felt 62 is broken and removed, then evaporative cooling commences.

While it is preferred that slot 32 or slit 60 is open to the perimeter thereof, slot 32 or slit 60 may be closed along the perimeter, and open only centrally.

All parts of the injectors or instrument 56 may be reconnectably disconnectable. Further, all parts of the injectors or instrument 56 may be disposable, or conversely, be reusable.

A variation of the injectors may comprise a blood-collecting capsule 24, wherein capsule 24 is evacuated to a negative pressure. The cold pad 34 desensitizes the skin over the vein. When needle 26 penetrates into the vein, blood will automatically fill capsule 24. Capsule 24 may comprise an elastomeric bulb, a glass tube, and so on. Similarly, capsule 24 may comprise a room-pressure capillary tube, such as for use in blood sugar monitoring.

The injectors may be resized so that larger volumes of fluids may be injected accordingly.

The injectors and instrument 56 may be connected to a vibrating means to further enhance tissue desensitization. Vibration is known to desensitize tissues.

The convenience and effectiveness of the injectors and instrument 56 facilitate the use of several useful oral injections currently underutilized by clinicians, as described in the following examples. These underutilized injections have the potential of substantially reducing the wait time between anesthetic administration and the effective anesthesia of tissues.

They are underutilized because standard injection techniques require excessive time to inject comfortably, or are excessively uncomfortable when administered within an efficient time period. In contrast, when the corresponding instrument 56 method is substituted, the teeth or tissues can be sufficiently anesthetized using a local anesthetic in conjunction with the injectors and instrument 56 so that a dental procedure can commence within about two minutes after initiating the injection, rather than the 3 to 10 minutes typically required for most of the various injections.

For a first example, to anesthetize the palatal tissue beside a single maxillary tooth using standard methods, a comfortable infiltration is performed in the buccal vestibule. After waiting about 4 minutes for the anesthesia to infiltrate to the buccal attached gingiva, a comfortable injection may be made through the papillae and into the palate. Then additional anesthetic is comfortably added to the palate for sufficient anesthesia. Anesthetizing the palate beside a maxillary tooth with this method requires about 10 minutes. More commonly, pressure is applied with a blunt mirror handle to the tissue and a needle is very uncomfortably inserted beside the blunt handle and completed in 45 seconds. With the injectors or instrument 56, the palate is comfortably anesthetized in 45-60 seconds.

For a second example, to anesthetize buccal attached gingiva of lower molars in preparation for administering an intraosseous injection, the standard method is to infiltrate anesthetic into the vestibule. After about 4 minutes, an additional comfortable injection is made into the buccal attached gingiva. Then a comfortable intraosseous injection can be initiated. Intraosseous anesthesia with this method requires about 8 minutes. More commonly, comfortable inferior alveolar nerve blocks are given instead, which require about 8 minutes for the teeth to become anesthetized. With the injectors or instrument 56, the buccal attached gingiva is comfortably anesthetized in 30 seconds, and an intraosseous injection comfortably anesthetizes the teeth in 2 additional minutes.

For a third example, the standard method for the anterior middle superior alveolar (AMSA) injection is to apply pressure to the palate with a blunt mirror handle, then uncomfortably inject beside the blunt handle between the first and second premolars. The injection continues about 1 minute. All the teeth and gingiva in the quadrant anterior to the site are anesthetized. Despite the discomfort, AMSA is used occasionally because of it's utility. When the injectors or instrument 56 is used in lieu of the blunt handle, the same injection is performed comfortably.

For a fourth example, the standard method for the nasopalatine injection is to apply pressure at the incisive papilla with a blunt mirror handle, then uncomfortably inject beside the blunt handle. The injection continues about 1 minute. This injection anesthetizes the area from teeth #6-11. Despite the discomfort, nasopalatine is used occasionally because of it's utility. When the injectors or instrument 56 is used in lieu of the blunt handle, the same injection is performed comfortably.

For a fifth example, palatal infiltrations of individual maxillary teeth from #4 to 13 are virtually unused clinically to anesthetize teeth for restorative dentistry. A standard method would be to apply pressure to the palate with a blunt mirror handle opposite the apex of a tooth, then uncomfortably inject beside the blunt handle, continuing for about 1 Minute. When the injectors or instrument 56 is used in lieu of the blunt handle, the same injection is performed comfortably. The injection is useful for augmenting anesthesia of teeth #4-13 in cases when buccal-facial infiltrations are found to be insufficient. Initial anesthesia of maxillary molars may be achieved using similar palatal infiltrations with the injectors or instrument 56. The palatal infiltrations are combined with buccal infiltrations to avoid posterior superior nerve blocks. The method may also be used to augment ineffective posterior superior nerve blocks for maxillary molars.

For a sixth example, the standard method for periodontal ligament (PDL) injections is to uncomfortably insert a needle into the PDL at two sites about a tooth. The sites are generally not desensitized prior to needle insertion. The injection time is about 90 seconds per site. The injectors may be used to comfortably pre-anesthetize the site before a PDL injection. Alternatively, a needle sharp 66 may be comfortably inserted directly into the PDL through a slit 60 of instrument 56. The injection time remains about 90 seconds per site with the use of the injectors or instrument 56.

Besides use with injections, the injectors or instrument 56 may be useful for other sharps procedures such as minor biopsies, removal of small bone fragments from the tissues, minor incision and drainages, and so on. The injectors or instrument 56 may be used to reduce irritation of laser tissue ablations and incisions. The injectors or instrument 56 may also be used to desensitize tissues of extraoral locations.

The drawings show a selection of capsule configurations capable of delaying the automatic flow of pressurized fluid from needle 26 until after needle 26 is sealingly inserted into the tissues. There are a number of other configurations and combinations that are effective for delaying injector fluid flow that are not shown. Such configurations are considered to be within the scope of the invention. Thus the scope of the invention should be determined by the appended claims and their legal equivalents, rather than by the examples given.

I claim:

1. An automatic injector having a tissue desensitizing, coolant-absorbent surface that substantially encompasses a needle protrusion area, and having a protrudable needle in an initial nonprotruded position, and having an unblockable fluid blocking means that initially blocks said fluid from flowing through said, needle and having a fluid container that contains and pressurizes said fluid to form a pressurized fluid, such as when pressure is exerted against said injector by pressing said injector against the tissues, wherein said needle is repositionable to a protruded position by pressure against said injector, and wherein said protruded position is repositioned a sufficient distance from said initial non-protruded position such that said fluid blocking means is unblocked to permit automatic fluid flow only after said needle is sealingly penetrated into the tissues.

2. A desensitizing injection method comprising the steps of: desensitizingly pressing a cooled, absorbent surface of an automatic, needle-containing, fluid-containing, injector onto the tissues, thereby pressurizing said fluid to form a pressurized fluid, wherein said pressurized fluid is initially blocked from flowing through said needle, protruding said needle from said injector such that said needle sealingly penetrates the tissues, and unblocking said pressurized fluid such that said pressurized fluid automatically flows through said needle and into the tissues
A desensitizing tissue puncture method comprising the steps of: unobstructively cooling a tissue puncture point by substantially encompassing said puncture point with a substantially unobstructive, cold, coolant-absorbent instrument, wherein said instrument substantially does not block visual or sharps access to said puncture point, puncturing said puncture point with a sharp, removing said instrument from the tissue, and withdrawing said sharp from said puncture point.
